SEQUENCE LISTING (88 sequences)

SEQ ID NO: 1 - protein, 227 aa, Homo sapiens
PVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 2 - protein, 219 aa, Homo sapiens
MGMSSLKLLKYVLFFFNLLFWICGCCILGFGIYLLIHNNFGVLFHNLPSLTLGNVFVIVGSIIMVVAFLGCMGSIKENKCLLMSFFILLLIILLAEVTLAILLFVYEQKLNEYVAKGLTDSIHRYHSDNSTKAAWDSIQSFLQCCGINGTSDWTSGPPASCPSDRKVEGCYAKARLWFHSNFLYIGIITICVCVIEVLGMSFALTLNCQIDKTSQTIGL

SEQ ID NO: 3 - protein, 237 aa, Homo sapiens
AVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 4 - protein, 236 aa, Homo sapiens
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 5 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 6 - protein, 547 aa, Homo sapiens
MATMVPSVLWPRACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYRLPERVELAPLPPWQPVGQNFTLRCQVEDGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

SEQ ID NO: 7 - protein, 427 aa, Homo sapiens
MFFTCGPNEAMVVSGFCRSPPVMVAGGRVFVLPCIQQIQRISLNTLTLNVKSEKVYTRHGVPISVTGIAQVKIQGQNKEMLAAACQMFLGKTEAEIAHIALETLEGHQRAIMAHMTVEEIYKDRQKFSEQVFKVASSDLVNMGISVVSYTLKDIHDDQDYLHSLGKARTAQVQKDARIGEAEAKRDAGIREAKAKQEKVSAQYLSEIEMAKAQRDYELKKAAYDIEVNTRRAQADLAYQLQVAKTKQQIEEQRVQVQVVERAQQVAVQEQEIARREKELEARVRKPAEAERYKLERLAEAEKSQLIMQAEAEAASVRMRGEAEAFAIGARARAEAEQMAKKAEAFQLYQEAAQLDMLLEKLPQVAEEISGPLTSANKITLVSSGSGTMGAAKVTGEVLDILTRLPESVERLTGVSISQVNHKPLRTA

SEQ ID NO: 8 - protein, 427 aa, Homo sapiens
GNCHTVGPNEALVVSGGCCGSDYKQYVFGGWAWAWWCISDTQRISLEIMTLQPRCEDVETAEGVALTVTGVAQVKIMTEKELLAVACEQFLGKNVQDIKNVVLQTLEGHLRSILGTLTVEQIYQDRDQFAKLVREVAAPDVGRMGIEILSFTIKDVYDKVDYLSSLGKTQTAVVQRDADIGVAEAERDAGIREAECKKEMLDVKFMADTKIADSKRAFELQKSAFSEEVNIKTAEAQLAYELQGAREQQKIRQEEIEIEVVQRKKQIAVEAQEILRTDKELIATVRRPAEAEAHRIQQIAEGEKVKQVLLAQAEAEKIRKIGEAEAAVIEAMGKAEAERMKLKAEAYQKYGDAAKMALVLEALPQIAAKIAAPLTKVDEIVVLSGDNSKVTSEVNRLLAELPASVHALTGVDLSKIPLIKKATGVQV

SEQ ID NO: 9 - protein, 1032 aa, Homo sapiens [sequence omitted]

SEQ ID NO: 10 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 11 - protein, 865 aa, Homo sapiens
MALVLGSLLLLGLCGNSFSGGQPSSTDAPKAWNYELPATNYETQDSHKAGPIGILFELVHIFLYVVQPRDFPEDTLRKFLQKAYESKIDYDKPETVILGLKIVYYEAGIILCCVLGLLFIILMPLVGYFFCMCRCCNKCGGEMHQRQKENGPFLRKCFAISLLVICIIISIGIFYGFVANHQVRTRIKRSRKLADSNFKDLRTLLNETPEQIKYILAQYNTTKDKAFTDLNSINSVLGGGILDRLRPNIIPVLDEIKSMATAIKETKEALENMNSTLKSLHQQSTQLSSSLTSVKTSLRSSLNDPLCLVHPSSETCNSIRLSLSQLNSNPELRQLPPVDAELDNVNNVLRTDLDGLVQQGYQSLNDIPDRVQRQTTTVVAGIKRVLNSIGSDIDNVTQRLPIQDILSAFSVYVNNTESYIHRNLPTLEEYDSYWWLGGLVICSLLTLIVIFYYLGLLCGVCGYDRHATPTTRGCVSNTGGVFLMVGVGLSFLFCWILMIIVVLTFVFGANVEKLICEPYTSKELFRVLDTPYLLNEDWEYYLSGKLFNKSKMKLTFEQVYSDCKKNRGTYGTLHLQNSFNISEHLNINEHTGSISSELESLKVNLNIFLLGAAGRKNLQDFAACGIDRMNYDSYLAQTGKSPAGVNLLSFAYDLEAKANSLPPGNLRNSLKRDAQTIKTIHQQRVLPIEQSLSTLYQSVKILQRTGNGLLERVTRILASLDFAQNFITNNTSSVIIEETKKYGRTIIGYFEHYLQWIEFSISEKVASCKPVATALDTAVDVFLCSYIIDPLNLFWFGIGKATVFLLPALIFAVKLAKYYRRMDSEDVYDDVETIPMKNMENGNNGYHKDHVYGIHNPVMTSPSQH

SEQ ID NO: 12 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 13 - protein, 150 aa, Homo sapiens [sequence omitted]

SEQ ID NO: 14 - protein, 867 aa, Homo sapiens
ATFISVQLKKTSEVDLAKPLVKFIQQTYPSGGEEQAQYCRAAEELSKLRRAAVGRPLDKHEGALETLLRYYDQICSIEPKFPFSENQICLTFTWKDAFDKGSLFGGSVKLALASLGYEKSCVLFNCAALASQIAAEQNLDNDEGLKIAAKHYQFASGAFLHIKETVLSALSREPTVDISPDTVGTLSLIMLAQAQEVFFLKATRDKMKDAIIAKLANQAADYFGDAFKQCQYKDTLPKEVFPVLAAKHCIMQANAEYHQSILAKQQKKFGEEIARLQHAAELIKTVASRYDEYVNVKDFSDKINRALAAAKKDNDFIYHDRVPDLKDLDPIGKATLVKSTPVNVPISQKFTDLFEKMVPVSVQQSLAAYNQRKADLVNRSIAQMREATTLANGVLASLNLPAAIEDVSGDTVPQSILTKSRSVIEQGGIQTVDQLIKELPELLQRNREILDESLRLLDEEEATDNDLRAKFKERWQRTPSNELYKPLRAEGTNFRTVLDKAVQADGQVKECYQSHRDTIVLLCKPEPELNAAIPSANPAKTMQGSEVVNVLKSLLSNLDEVKKEREGLENDLKSVNFDMTSKFLTALAQDGVINEEALSVTELDRVYGGLTTKVQESLKKQEGLLKNIQVSHQEFSKMKQSNNEANLREEVLKNLATAYDNFVELVANLKEGTKFYNELTEILVRFQNKCSDIVFARKTERDELLKDLQQSIAREPSAPSIPTPAYQSSPAGGHAPTPPTPAPRTMPPTKPQPPARPPPPVLPANRAPSATAPSPVGAGTAAPAPSQTPGSAPPPQAQGPPYPTYPGYPGYCQMPMPMGYNPYAYGQYNMPYPPVYHQSPGQAPYPGPQQPSYPFPQPPQQSYYPQQ

SEQ ID NO: 15 - protein, 297 aa, Homo sapiens
SLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQGIREVILCKDQDGKIGLRLKSIDNGIFVQLVQANSPASLVGLRFGDQVLQINGENCAGWSSDKAHKVLKQAFGEKITMTIRDRPFERTITMHKDSTGHVGFIFKNGKITSIVKDSSAARNGLLTEHNICEINGQNVIGLKDSQIADILSTSGTVVTITIMPAFIFEHIIKRMAPSIMKSLMDHTIPEV

SEQ ID NO: 16 - protein, 292 aa, Homo sapiens
MSSLYPSLEDLKVDQAIQAQVRASPKMPALPVQATAISPPPVLYPNLAELENYMGLSLSSQEVQESLLQIPEGDSTAVSGPGPGQMVAPVTGYSLGVRRAEIKPGVREIHLCKDERGKTGLRLRKVDQGLFVQLVQANTPASLVGLRFGDQLLQIDGRDCAGWSSHKAHQVVKKASGDKIVVVVRDRPFQRTVTMHKDSMGHVGFVIKKGKIVSLVKGSSAARNGLLTNHYVCEVDGQNVIGLKDKKIMEILATAGNVVTLTIIPSVIYEHMVKKLPPVLLHHTMDHSIPDA

SEQ ID NO: 17 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

SEQ ID NO: 18 - protein, 33 aa, Saccharomyces cerevisiae
RMKQLEDKVEELLSKNYHLENEVARLKKLVGER

SEQ ID NO: 19 - protein, 38 aa, Saccharomyces cerevisiae
CGGREGVLKKLRAVENELHYNKSLLEEVKDELQKMRQL

SEQ ID NO: 20 - protein, 30 aa, Bacteriophage T4
GYIPEAPRDGQAYVRKDGEWVFLSTFLSPA

SEQ ID NO: 21 - protein, 60 aa, respiratory syncytial virus
ETIETFDNNEEESSYSYEEINDQTNDNITARLDRIDEKLSEILGMLHTLVVASAGPTSAR

SEQ ID NO: 22 - protein, 63 aa, Homo sapiens
TAFSNMDDMLQKAHLVIEGTFIYLRDSTEFFIRVRDGWKKLQLGELIPIPADSPPPPALSSNP

SEQ ID NO: 23 - protein, 29 aa, Homo sapiens
LEEKVKTLKAQNSELASTANMLREQVAQL

SEQ ID NO: 24 - protein, 21 aa, Homo sapiens
FINFCLILICLLLICIIVMLL

SEQ ID NO: 25 - protein, 34 aa, Homo sapiens
SVSEIQLMHNLGKHLNSMERVEWLRKKLQDVHNF

SEQ ID NO: 26 - protein, 40 aa, Homo sapiens
VQLAHHFSEPEITLIIFGVMAGVIGTILLISYGIRRLIKK

SEQ ID NO: 27 - protein, 60 aa, Human immunodeficiency virus
GAASITLTVQARRLLSGIVQQQNNLLRAIEAQQHLLKLTVWGIKQLQARILAVERYLKDQ

SEQ ID NO: 28 - protein, 56 aa, Human papillomavirus type 45
GSHMAEPQRHKILCVCCKCDGRIELTVESSAEDLRTLQQLFLSTLSFVCPWCATNQ

SEQ ID NO: 29 - protein, 153 aa, Homo sapiens
GLAAAIAGAKLRKVSKQEEASGGPTAPKAESGRSGGGGLMEEMNAMLARRRKATQVGEKTPKDESANQEEPEARVPAQSESVRRPWEKNSTTLPRMKSSSSVTTSETQPCTPSSSDYSDLQRVKQELLEEVKKELQKVKEEIIEAFVQELRKR

SEQ ID NO: 30 - protein, 918 aa, Homo sapiens [sequence omitted]

SEQ ID NO: 31 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 32 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 33 - protein, 502 aa, Homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 34 - protein, 791 aa, Homo sapiens
MPVPWFLLSLALGRSPVVLSLERLVGPQDATHCSPVSLEPWGDEERLRVQFLAQQSLSLAPVTAATARTALSGLSGADGRREERGRGKSWVCLSLGGSGNTEPQKKGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 35 - protein, 739 aa, Homo sapiens
MAPWLQLCSVFFTVNACLNGSQLAVAAGGSGRARGADTCGWRGVGPASRNSGLYNITFKYDNCTTYLNPVGKHVIADAQNITISQYACHDQVAVTILWSPGALGIEFLKGFRVILEELKSEGRQCQQLILKDPKQLNSSFKRTGMESQPFLNMKFETDYFVKVVPFPSIKNESNYHPFFFRTRACDLLLQPDNLACKPFWKPRNLNISQHGSDMQVSFDHAPHNFGFRFFYLHYKLKHEGPFKRKTCKQEQTTETTSCLLQNVSPGDYIIELVDDTNTTRKVMHYALKPVHSPWAGPIRAVAITVPLVVISAFATLFTVMCRKKQQENIYSHLDEESSESSTYTAALPRERLRPRPKVFLCYSSKDGQNHMNVVQCFAYFLQDFCGCEVALDLWEDFSLCREGQREWVIQKIHESQFIIVVCSKGMKYFVDKKNYKHKGGGRGSGKGELFLVAVSAIAEKLRQAKQSSSAALSKFIAVYFDYSCEGDVPGILDLSTKYRLMDNLPQLCSHLHSRDHGLQEPGQHTRQGSRRNYFRSKSGRSLYVAICNMHQFIDEEPDWFEKQFVPFHPPPLRYREPVLEKFDSGLVLNDVMCKPGPESDFCLKVEAAVLGATGPADSQHESQHGGLDQDGEARPALDGSAALQPLLHTVKAGSPSDMPRDSGIYDSSVPSSELSLPLMEGLSTDQTETSSLTESVSSSSGLGEEEPPALPSKLLSSGSCKADLGCRSYTDELHAVAPL

SEQ ID NO: 36 - protein, 667 aa, Homo sapiens
MGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 37 - protein, 629 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 38 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 39 - protein, 398 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 40 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 41 - protein, 862 aa, Homo sapiens
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 42 - protein, 11 aa, Vesicular stomatitis virus
YTDIEMNRLGK

SEQ ID NO: 43 - protein, 511 aa, Vesicular stomatitis virus
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 44 - protein, 17 aa, Homo sapiens
CDVEWVDVSSLEWDLPC

SEQ ID NO: 45 - protein, 12 aa, Homo sapiens
DITWDQLWDLMK

SEQ ID NO: 46 - protein, 12 aa, Human immunodeficiency virus
GRKKRRQRRRPQ

SEQ ID NO: 47 - protein, 222 aa, Haemophilus influenzae
GLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 48 - protein, 338 aa, Artificial sequence
  Fusion polypeptide comprising at least one human sequence fused to at least one sequence deriving from any other species
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQGTEDSGTTVLLPLVIFFGLCLLSLLFIGLMYRYQRWKGTGYIPEAPRDGQAYVRKDGEWVFLSTFLSPANGGGGSGRSLYPSLEDLKVDKVIQAQTAFSANPANPAILSEASAPIPHDGNLYPRLYPELSQYMGLSLEGG

SEQ ID NO: 49 - protein, 833 aa, Artificial sequence
  Fusion polypeptide comprising at least one human sequence fused to at least one sequence deriving from any other species
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQRMKQLEDKVEELLSKNYHLENEVARLKKLVGERGSGSGSGSGSSLYPSLEDLKVDKVIQAQTAYSANPASQAFVLVDASAALPPDGNLYPKLYPELSQYMGLSLNEAEICESMPMVSGAPAQGQLVARPSSVNYMVAPVTGNDAGIRRAEIK

SEQ ID NO: 50 - protein, 859 aa, Artificial sequence
  Fusion polypeptide comprising at least one human sequence fused to at least one sequence deriving from any other species
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDETIETFDNNEEESSYSYEEINDQTNDNITARLDRIDEKLSEILGMLHTLVVASAGPTSARGSGSGSGSGSSLYPSLEDLKVDKVIQAQTAYSANPASQAFVLVDASAALPPDGNLYPKLYPELSQYMGLSLNEAEICESMPMVSGAPAQGQLVARPSSVNYMVAPVTGNDAGIRRAEIK

SEQ ID NO: 51 - protein, 782 aa, Artificial sequence
  Fusion polypeptide comprising at least one human sequence fused to at least one sequence deriving from any other species
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDRMKQLEDKVEELLSKNYHLENEVARLKKLVGERGSGSGSGSGSMDQARSAFSNLFGGEPLSYTRFSLARQVDGDNSHVEMKLAVDEEENADNNTKANVTKPK

SEQ ID NO: 52 - protein, 886 aa, Artificial sequence
  Fusion polypeptide comprising at least one human sequence fused to at least one sequence deriving from any other species
MLTLQTWLVQALFIFLTTESTGCDVEWVDVSSLEWDLPCGSGSGSGSGSELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDETIETFDNNEEESSYSYEEINDQTNDNITARLDRIDEKLSEILGMLHTLVVASAGPTSARGSGSGSGSGSSLYPSLEDLKVDKVIQAQTAYSANPASQAFVLVDASAALPPDGNLYPKLYPELSQYMGLSLNEAEICESMPMVSGAPAQGQLVARPSSVNYMVAPVTGNDAGIRRAEIK

SEQ ID NO: 53 - protein, 847 aa, Artificial sequence
  Fusion polypeptide comprising at least one human sequence fused to at least one sequence deriving from any other species
METIETFDNNEEESSYSYEEINDQTNDNITARLDRIDEKLSEILGMLHTLVVASAGPTSARMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECEGLAGTESPGGEEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEFGSGSGSGSGSCDVEWVDVSSLEWDLPC

SEQ ID NO: 54 - protein, 859 aa, Artificial sequence
  Fusion polypeptide comprising at least one human sequence fused to at least one sequence deriving from any other species
MLTLQTWLVQALFIFLTTESTGCDVEWVDVSSLEWDLPCGSGSGSGSGSELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDRMKQLEDKVEELLSKNYHLENEVARLKKLVGERGSGSGSGSGSSLYPSLEDLKVDKVIQAQTAYSANPASQAFVLVDASAALPPDGNLYPKLYPELSQYMGLSLNEAEICESMPMVSGAPAQGQLVARPSSVNYMVAPVTGNDAGIRRAEIK

SEQ ID NO: 55 - protein, 449 aa, Artificial sequence
  Fusion polypeptide comprising at least one human sequence fused to at least one sequence deriving from any other species
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLNGGGGSGRGYIPEAPRDGQAYVRKDGEWVFLSTFLSPANGGGGSGRDNKFNKEQQNAFYEILHLPNLNEEQRNAFIQSLKDDPSQSANLLAEAKKLNDAQAPKPQGTEDSGTTVLLPLVIFFGLCLLSLLFIGLMYRYQRWKGTNGGGGSGRGYIPEAPRDGQAYVRKDGEWVFLSTFLSPANGGGGSGRSLYPSLEDLKVDKVIQAQTAFSANPANPAILSEASAPIPHDGNLYPRLYPELSQYMGLSLEGG

SEQ ID NO: 56 - protein, 608 aa, Artificial sequence
  Fusion polypeptide comprising at least one human sequence fused to at least one sequence deriving from any other species
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENGSGSGSGSGSEEKRRIRRERNKMAAAKCRNRRRELTDTLQAETDQLEDEKSALQTEIANLLKEKEKLEFILAAHGSGSGSGSGSSLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQ

SEQ ID NO: 57 - protein, 805 aa, Artificial sequence
  Fusion polypeptide comprising at least one human sequence fused to at least one sequence deriving from any other species
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASGSGSGSGSGSRIKAERKRMRNRIAASKCRKRKLERIARLEEKVKTLKAQNSELASTANMLREQVAQLKQKVMNHGSGSGSGSGSSLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQ

SEQ ID NO: 58 - protein, 752 aa, Artificial sequence
  Fusion polypeptide comprising at least one human sequence fused to at least one sequence deriving from any other species
MPVPWFLLSLALGRSPVVLSLERLVGPQDATHCSPVSLEPWGDEERLRVQFLAQQSLSLAPVTAATARTALSGLSGADGRREERGRGKSWVCLSLGGSGNTEPQKKGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLRMKQLEDKVEELLSKNYHLENEVARLKKLVGERGSGSGSGSGSSLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQ

SEQ ID NO: 59 - protein, 275 aa, Artificial sequence
  Fusion polypeptide comprising at least one human sequence fused to at least one sequence deriving from any other species
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDTQGSLNSLSGESGELEEPIESNEIDLTIDSDLRPKSSWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 60 - protein, 359 aa, Artificial sequence
  Fusion polypeptide comprising at least one human sequence fused to at least one sequence deriving from any other species
MKWVTFISLLFLFSSAYSPEENEPAQTLPALKPRENDLDSWELEKESPVASWSGPALQEPDGDELSESSLSTSELGAMKKHKGGLLRKGAKLFFRRRHQQKDPGLSQSHNDLVGSGSGSGSGSMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 61 - protein, 313 aa, Artificial sequence
  Fusion polypeptide comprising at least one human sequence fused to at least one sequence deriving from any other species
MSAPRIWLAQALLFFLTTESIGPAMASHKDDEEKFFPQSGVLIFGKQGSEKTNVDIEKVMITDEEEIRTTNPVATEQYGSVSTNLQRGNRYTLSQGWQAATADVNTQGVLPGMVWQDRDTPKFAQGEIEAIVVPVCLAFLLTTLLGVLFCFNKRDLIKKHIRMKQLEDKVEELLSKNYHLENEVARLKKLVGERGSGSGSGSGSSLYPSLEDLKVDKVIQAQTAYSANPASQAFVLVDASAALPPDGNLYPKLYPELSQYMGLSLNEAEICESMPMVSGAPAQGQLVARPSSVNYMVAPVTGNDAGIRRAEIK

SEQ ID NO: 62 - protein, 313 aa, Artificial sequence
  Fusion polypeptide comprising at least one human sequence fused to at least one sequence deriving from any other species
MSAPRIWLAQALLFFLTTESIGPAMASHKDDEEKFFPQSGVLIFGKQGSEKTNVDIEKVMITDEEEIRTTNPVATEQYGSVSTNLQRGNRTLAVPFKQAATADVNTQGVLPGMVWQDRDTPKFAQGEIEAIVVPVCLAFLLTTLLGVLFCFNKRDLIKKHIRMKQLEDKVEELLSKNYHLENEVARLKKLVGERGSGSGSGSGSSLYPSLEDLKVDKVIQAQTAYSANPASQAFVLVDASAALPPDGNLYPKLYPELSQYMGLSLNEAEICESMPMVSGAPAQGQLVARPSSVNYMVAPVTGNDAGIRRAEIK

SEQ ID NO: 63 - protein, 253 aa, Artificial sequence
  Fusion polypeptide comprising at least one human sequence fused to at least one sequence deriving from any other species
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMQRGNRTLAVPFKQAATANRVPDSCCINVTVGCGINFNEKAIHKEGCVEKIGGWLRKNVLVVAAAALGIAFVEVLGIVFACCLVKSIRSGYEVM

SEQ ID NO: 64 - protein, 253 aa, Artificial sequence
  Fusion polypeptide comprising at least one human sequence fused to at least one sequence deriving from any other species
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMQRGNRYTLSQGWQAATANRVPDSCCINVTVGCGINFNEKAIHKEGCVEKIGGWLRKNVLVVAAAALGIAFVEVLGIVFACCLVKSIRSGYEVM

SEQ ID NO: 65 - protein, 267 aa, Artificial sequence
  Fusion polypeptide comprising at least one human sequence fused to at least one sequence deriving from any other species
MRMKQLEDKVEELLSKNYHLENEVARLKKLVGERMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECEGLAGTESPGGEEPGEDFPAARRHHHHHHGSGSGSGSGSPAMASHKDDEEKFFPQSGVLIFGKQGSEKTNVDIEKVMITDEEEIRTTNPVATEQYGSVSTNLQRGNRTLAVPFKQAATADVNTQGVLPGMVWQDRD

SEQ ID NO: 66 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 67 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 68 - protein, 514 aa, Artificial sequence
  Fusion polypeptide comprising at least one human sequence fused to at least one sequence deriving from any other species
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTRMKQLEDKVEELLSKNYHLENEVARLKKLVGERGSGSGSGSGSSLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQ

SEQ ID NO: 69 - protein, 29 aa, Homo sapiens
LQAETDQLEDEKSALQTEIANLLKEKEKL

SEQ ID NO: 70 - protein, 574 aa, Homo sapiens
MRTLLTILTVGSLAAHAPEDPSDLLQHVKFQSSNFENILTWDSGPEGTPDTVYSIEYKTYGERDWVAKKGCQRITRKSCNLTVETGNLTELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTWTYSFSGAFLFSMGFLVAVLCYLSYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSRPCSPSDQGPSPWGLLESLVCPKDEAKSPAPETSDLEQPTELDSLFRGLALTVQWES

SEQ ID NO: 71 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 72 - protein, 310 aa, Homo sapiens; identical to SEQ ID NO: 12

SEQ ID NO: 73 - protein, 201 aa, Homo sapiens
MRRAWILLTLGLVACVSAESRAELTSDKDMYLDNSSIEEASGVYPIDDDDYASASGSGADEDVESPELTTSRPLPKILLTSAAPKVETTTLNIQNKIPAQTKSPEETDKEKVHLSDSERKMDPAEEDTNVYTEKHSDSLFKRTEVLAAVIAGGVIGFLFAIFLILLLVYRMRKKDEGSYDLGERKPSSAAYQKAPTKEFYA

SEQ ID NO: 74 - protein, 442 aa, Homo sapiens
MKPGPPHRAGAAHGAGAGAGAAAGPGARGLLLPPLLLLLLAGRAAGAQRWRSENFERPVDLEGSGDDDSFPDDELDDLYSGSGSGYFEQESGIETAMRFSPDVALAVSTTPAVLPTTNIQPVGTPFEELPSERPTLEPATSPLVVTEVPEEPSQRATTVSTTMATTAATSTGDPTVATVPATVATATPSTPAAPPFTATTAVIRTTGVRRLLPLPLTTVATARATTPEAPSPPTTAAVLDTEAPTPRLVSTATSRPRALPRPATTQEPDIPERSTLPLGTTAPGPTEVAQTPTPETFLTTIRDEPEVPVSGGPSGDFELPEEETTQPDTANEVVAVGGAAAKASSPPGTLPKGARPGPGLLDNAIDSGSSAAQLPQKSILERKEVLVAVIVGGVVGALFAAFLVTLLIYRMKKKDEGSYTLEEPKQASVTYQKPDKQEEFYA

SEQ ID NO: 75 - protein, 198 aa, Homo sapiens
MAPARLFALLLFFVGGVAESIRETEVIDPQDLLEGRYFSGALPDDEDVVGPGQESDDFELSGSGDLDDLEDSMIGPEVVHPLVPLDNHIPERAGSGSQVPTEPKKLEENEVIPKRISPVEESEDVSNKVSMSSTVQGSNIFERTEVLAALIVGGIVGILFAVFLILLLMYRMKKKDEGSYDLGKKPIYKKAPTNEFYA

SEQ ID NO: 76 - protein, 59 aa, Homo sapiens
MDQARSAFSNLFGGEPLSYTRFSLARQVDGDNSHVEMKLAVDEEENADNNTKANVTKPK

SEQ ID NO: 77 - protein, 267 aa, Homo sapiens
MGSACIKVTKYFLFLFNLIFFILGAVILGFGVWILADKSSFISVLQTSSSSLRMGAYVFIGVGAVTMLMGFLGCIGAVNEVRCLLGLYFAFLLLILIAQVTAGALFYFNMGKLKQEMGGIVTELIRDYNSSREDSLQDAWDYVQAQVKCCGWVSFYNWTDNAELMNRPEVTYPCSCEVKGEEDNSLSVRKGFCEAPGNRTQSGNHPEDWPVYQEGCMEKVQAWLQENLGIILGVGVGVAIIELLGMVLSICLCRHVHSEDYSKVPKY

SEQ ID NO: 78 - protein, 387 aa, Homo sapiens
MPRPRLLAALCGALLCAPSLLVALDICSKNPCHNGGLCEEISQEVRGDVFPSYTCTCLKGYAGNHCETKCVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 79 - protein, 556 aa, Homo sapiens [sequence omitted]

SEQ ID NO: 80 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 81 - protein, 620 aa, Homo sapiens
MQVSKRMLAGGVRSMPSPLLACWQPILLLVLGSVLSGSATGCPPRCECSAQDRAVLCHRKRFVAVPEGIPTETRLLDLGKNRIKTLNQDEFASFPHLEELELNENIVSAVEPGAFNNLFNLRTLGLRSNRLKLIPLGVFTGLSNLTKLDISENKIVILLDYMFQDLYNLKSLEVGDNDLVYISHRAFSGLNSLEQLTLEKCNLTSIPTEALSHLHGLIVLRLRHLNINAIRDYSFKRLYRLKVLEISHWPYLDTMTPNCLYGLNLTSLSITHCNLTAVPYLAVRHLVYLRFLNLSYNPISTIEGSMLHELLRLQEIQLVGGQLAVVEPYAFRGLNYLRVLNVSGNQLTTLEESVFHSVGNLETLILDSNPLACDCRLLWVFRRRWRLNFNRQQPTCATPEFVQGKEFKDFPDVLLPNYFTCRRARIRDRKAQQVFVDEGHTVQFVCRADGDPPPAILWLSPRKHLVSAKSNGRLTVFPDGTLEVRYAQVQDNGTYLCIAANAGGNDSMPAHLHVRSYSPDWPHQPNKTFAFISNQPGEGEANSTRATVPFPFDIKTLIIATTMGFISFLGVVLFCLVLLFLWSRGKGNTKHNIEIEYVPRKSDAGISSADAPRKFNMKMI

SEQ ID NO: 82 - protein, 20 aa, Haemophilus influenzae
GLFGAIAGFIENGWEGMIDG

SEQ ID NO: 83 - protein, 389 aa, Homo sapiens
AVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 84 - protein, 29 aa, Rabies virus
YTIWMPENPRPGTPCDIFTNSRGKRASNG

SEQ ID NO: 85 - protein, 100 aa, Homo sapiens
MPFAEDKTYKYICRNFSNFCNVDVVEILPYLPCLTARDQDRLRATCTLSGNRDTLWHLFNTLQRRPGWVEYFIAALRGCELVDLADEVASVYQSYQPRTS

SEQ ID NO: 86 - protein, 59 aa, Human immunodeficiency virus type 1
LISEEDLLQSRPEPTAPPEESFRSGVETTTPPQKQEPIDKELYPLTSLRSLFGNDPSSQ

SEQ ID NO: 87 - protein, 444 aa, Artificial sequence
  Fusion polypeptide comprising at least one human sequence fused to at least one sequence deriving from any other species
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLNGGGGSGRGYIPEAPRDGQAYVRKDGEWVFLSTFLSPANGGGGSGRDNKFNKEQQNAFYEILHLPNLNEEQRNAFIQSLKDDPSQSANLLAEAKKLNDAQAPKPQGTEDSGTTVLLPLVIFFGLCLLSLLFIGLMYRYQRWKGTNGGGGSGRGYIPEAPRDGQAYVRKDGEWVFLSTFLSPANGGGGSGLISEEDLLQSRPEPTAPPEESFRSGVETTTPPQKQEPIDKELYPLTSLRSLFGNDPSSQ

SEQ ID NO: 88 - protein, 570 aa, Artificial sequence
  Fusion polypeptide comprising at least one human sequence fused to at least one sequence deriving from any other species
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYGGGGSGGGGSGGGGSLISEEDLLQSRPEPTAPPEESFRSGVETTTPPQKQEPIDKELYPLTSLRSLFGNDPSSQ

(19) United States
(12) Patent Application Publication
(10) Pub. No.: US 2020/0062813 A1
(43) Pub. Date: Feb. 27, 2020

(54) Improved Loading of EVs with Therapeutic Proteins

(71) Applicant: Evox Therapeutics Ltd, Oxford (GB)

(72) Inventors: Joel NORDIN, Stockholm (SE); Dhanu GUPTA, Stockholm (SE)

(21) Appl. No.: 16/487,667

(22) PCT Filed: Jan. 19, 2018
(86) PCT No.: PCT/EP2018/051359

§ 371 Date: Aug. 21, 2019

(30) Foreign Application Priority Data
Feb. 22, 2017 (GB) 1702863.0

(51) Int. Cl. C07K 14/47

(52) U.S. Cl. CPC C07K 14/4718; C07K 2319/055; A61K 45/06; C07K 2319/73; C07K 2319/06

(57) ABSTRACT

The present invention pertains to improved methods for loading of extracellular vesicles (EVs), such as exosomes, with various types of proteins of interest. More specifically, the invention relates to loading of EVs using fusion polypeptide constructs, as well as i.a. fusion constructs per se and EVs carrying such fusion polypeptides. The design of the fusion polypeptides is key to enable both efficient surface-display and internal loading into EVs of proteins of interest.

### TECHNICAL FIELD

The present invention pertains to improved methods for loading of extracellular vesicles (EVs) with various types of proteins of interest. More specifically, the invention relates to loading of EVs using fusion construct comprising multimerization domain(s), as well as i.a. fusion constructs per se and EVs carrying such fusion constructs.

### BACKGROUND ART

Extracellular vesicles (EVs) are gaining increasing attention for their utility as delivery vehicles for a variety of therapeutic modalities ranging from small molecule agents and RNA therapeutics to antibodies and other protein biologics. Delivery of polypeptide-based agents with the aid of exosomes is disclosed in the seminal patent application WO2013/084000, which describes how polypeptide-based therapeutics may be loaded into exosomes via both exogenous and endogenous loading techniques. Exogenous loading of exosomes is in WO2013/084000 carried out using electroporation or transfection of the polypeptide of interest into exosomes post-isolation from the parental cell, whereas endogenous loading is based on transfection of the parental cell with a construct encoding the polypeptide of interest, followed by overexpression of the construct and harvesting of exosomes comprising the biotherapeutic polypeptide.

Another groundbreaking patent application (WO2014/168548) discloses therapeutic delivery vesicles, such as exosomes, having attached to their membrane a polypeptide construct comprising at least one carrier polypeptide fused to at least one therapeutic polypeptide, which is present at least partially on the outside of the vesicle, so that it is displayed to the extravesicular environment. Other patent applications have attempted to use exosomes for the delivery of protein biologics, such as, in the case of WO2015/138878, heparin-binding epidermal growth factor (HB-EGF).

WO2014/168548, in particular, represents an excellent example of how genetic modification of parental EV-producing cells enables production of EVs, typically exosomes, displaying therapeutically relevant and highly active protein polypeptide-based biologics, with significant clinical utility. In two recent studies, Yim et al., Nature Communications, 7:12277, 2016) and Lainscek et al., BBRC, 2017), the authors show how two separate protein constructs can be dimerized in exosomes with the aid of optogenetic heterodimerization domains. These two studies represent excellent examples of how genetic engineering can be applied to specifically dimerize two protein constructs in exosomes, but both studies are completely silent as to how the loading efficiency of a single type of polypeptide construct can be improved through protein engineering. Thus, there is still significant room in the art for further optimizing the number of proteins of interest that are expressed in or on EVs after genetic modification of parental cells, and to increase the potency and/or the affinity and avidity of such therapeutic proteins of interest, for both targeting and therapeutic purposes.

### SUMMARY OF THE INVENTION

To overcome the problems in the art and to enable the development of highly potent EVs (typically exosomes) carrying proteins of interest of different kinds, the present invention discloses sophisticated fusion polypeptide constructs comprising at least one protein of interest (POI), at least one exosomal sorting domain, and at least one multimerization domain. Surprisingly, this inventive design results in orders of magnitude higher density of proteins of interest that can be loaded into or onto EVs, and equally surprisingly it also results in both increased EV yields and enhanced avidity of the protein of interest for its target (i.e. the total affinity of e.g. decoy receptors for their target molecule or the total affinity of targeting ligands for the receptor on a target cell or tissue). As above-mentioned, dimerization of two separate fusion protein constructs as a loading strategy for soluble proteins into EVs has been described by Yim et al. and by Lainscek et al. However, the present invention represents an entirely novel approach as it uses multimerization domains, in particular homo-multimerization domains, to enhance loading of a single type of fusion polypeptide constructs, i.e. multiple copies of one type of fusion protein which comprises an exosomal protein, the POI and the multimerization domain in a single polypeptide construct. Importantly, the present invention thus addresses a completely different problem, i.e. not the loading of soluble proteins as such but the improvement of already functioning loading and optionally surface display of a protein of interest.

In a first aspect, the present invention relates to fusion polypeptides comprising at least one POI, at least one exosomal sorting domain, and at least one multimerization domain. In preferred embodiments, the multimerization domains of the present invention induce homo-multimerization of a single type of fusion polypeptide construct into multimers comprising several such polypeptide constructs. Various multimerization domains have been evaluated and tested in vitro and in vivo, indicating that this strategy can be applied broadly by judicious genetic engineering of the polynucleotide constructs and vectors encoding for the fusion polypeptides.

The present invention is equally applicable to the loading of proteins of interest onto the surface of EVs as well as to the interior of the EVs, which provides for the creation both luminally loaded EV therapeutics as well as for the development of targeted EVs and EVs comprising surface-displayed therapeutic proteins (e.g. decoy receptors, transport proteins such as the NPC1 protein, the LAMP2 protein, GM2-activator protein, cystinosin, CLN3 or CLN6, mucolipin-1, G-protein coupled receptors, antibodies or single chain antibodies or fragments thereof, or essentially any transmembrane protein of interest). Importantly, the EVs may comprise more than one POI, e.g. a combination of (i) a targeting peptide/protein (i.e. a targeting POI) displayed on the surface, and (ii) a therapeutic POI (displayed either on the surface or inside the EV). A non-limiting example of a therapeutic POI that may be displayed on EV surfaces are so called decoy receptors, i.e. proteins that bind to and inhibit e.g. cytokines or other factors. A non-limiting example of luminally (internally) loaded POIs may include enzymes for e.g. enzyme replacement therapy or nucleases for binding to and modulating DNA.

In another aspect, the present invention relates to the fusion polypeptide-carrying EVs per se, i.e. EVs comprising fusion polypeptides as per the present invention. A single EV may as abovementioned comprise one or more different types of fusion polypeptide constructs, as well as multiple copies of each type of fusion polypeptide construct. In preferred embodiments, a single EV (such as an exosome) may comprise more than 50 copies of a desired fusion polypeptide (and thus more than 50 copies of the POI, regardless of the nature of the POI), preferably more than 75 copies of the fusion polypeptide and even more preferably more than 100 or even more than 300 copies of the fusion polypeptide per EV. Furthermore, the present invention also relates to EV source cells, as well as cells comprising both polynucleotide constructs and cells comprising the fusion polypeptides encoded for by the polynucleotide constructs.

In yet other aspects, the present invention pertains to compositions comprising a plurality of fusion polypeptide-carrying EVs as per the present invention. Typically, these compositions are pharmaceutical compositions for use in vivo, but compositions and formulations for use in vitro are also within the scope of the present invention.

In another aspect, the present invention relates to methods for loading POIs into EVs. Such methods may comprise the steps of: (i) providing a fusion polynucleotide construct that encodes for at least one multimerization domain, at least one exosomal sorting protein, and the POI, and (ii) expressing said fusion polynucleotide construct in an EV-producing cell to load the EV with the fusion polypeptide and hence with the POI. The cell for production of EVs may be a primary cell or a cell line, and the polynucleotide construct may be essentially any suitable type of construct from which expression of the fusion polypeptide can be carried out.

In further aspects, the instant invention relates to methods for their production and purification of EVs as per the present invention. Also, the EVs as per the present invention may be utilized in the prophylaxis and/or treatment of a large number of diseases and ailments, notably within cancer, inflammation and autoimmunity, neuroinflammatory and neurodegenerative disorders, genetic diseases, lysosomal storage disorders, organ injuries and failure, muscular dystrophies such as DMD, infectious diseases, etc.

### BRIEF DESCRIPTION OF THE FIGURES

FIG. 1 shows how the inclusion of a multimerization domain, fold-on, increases the loading and the effect of the EVs in a dose-dependent manner compared to EVs comprising fusion polypeptides without the multimerization domain.

FIG. 2 illustrates the increased activity in vitro of GP130 decoy receptor exosomes after the insertion of a multimerization domain.

FIG. 3 depicts the results of animal studies of LPS-induced systemic inflammation treated with GP130-GCN4 leucine zipper syntenin decoy EVs and TNFR1 foldon syndecan decoy EVs, comparing EVs comprising fusion polypeptides with multimerization domains (light gray circles and downward-pointing triangles, respectively) versus EVs comprising fusion polypeptides without multimerization domains (upward pointing triangles).

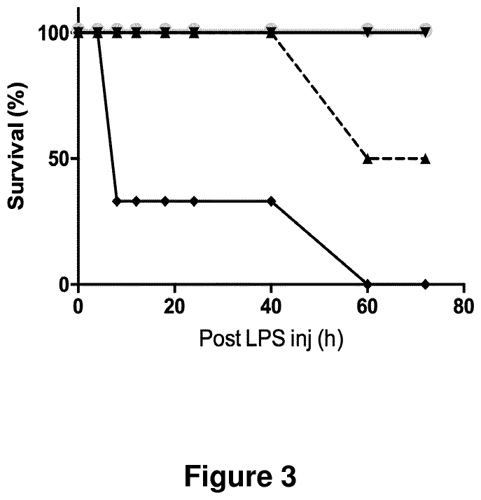

FIG. 4: NTA data showing increased particle release from cells stably expressing a multimerization domain together with an exosomal sorting domain.

FIG. 5 shows HeLa cells that stably express a reporter for IL6 activation was treated with hyper-IL6 and with EVs (obtained from bone marrow-derived mesenchymal stromal cells) equipped with a fusion protein construct comprising the gp130 decoy receptor as the POI, the 2G12 IgG homodimer domain as the multimerization domain, and ALIX as the exosomal sorting domain. Fusion proteins comprising the 2G12 IgG homodimer domain is clearly better than the EVs only equipped with GP130-ALIX at inhibiting IL6-mediated signaling, illustrating the importance of the multimerization domains to drive increased loading into EVs of fusion protein constructs.

FIG. 6 shows adipose tissue MSCs stably transduced with 4 different Gaussia reporter constructs. Gaussia was fused with CD63 and CD81 with and without multimerization domains. EVs were harvested from conditioned media (incubated for 48 h) and purified with tangential flow and Capto-core liquid chromatography columns. The Gaussia luciferase signal was measured on the harvested EVs as a measurement of CD63/81 loading. As can clearly be seen from figure X, the CD63/81 constructs with the cardiac phospholamban transmembrane pentamer domain has a higher signal and thus increased loading of the EV protein.

FIG. 7 depicts HeLa cells that stably express a reporter for IL6 activation was treated with hyper-IL6 and with EVs (obtained from bone marrow-derived mesenchymal stromal cells) equipped with a fusion protein construct comprising the gp130 decoy receptor as the POI, the leucine zipper homodomain as the multimerization domain, and various different exosomal sorting domain. Fusion proteins comprising the leucine zipper domain is clearly better at inhibiting IL6-mediated signaling than the EVs only equipped with GP130 fused to exosomal sorting domains, evidencing the importance of the multimerization domains and their applicability across virtually all types of exosomal polypeptides.

### DETAILED DESCRIPTION OF THE INVENTION

The present invention pertains to polypeptide constructs comprising at least three domains: (i) at least one protein of interest (POI), (ii) at least one multimerization domain, and (iii) at least one exosomal sorting domain. Further, the present invention also relates to EVs comprising such tri-domain polypeptide constructs, wherein the polypeptide construct is present either essentially in the lumen of the EV or in association (e.g. in) the EV membrane, on the outside or on the inside or both. Furthermore, the invention relates to various adjacent aspects as will be described in greater detail below, for instance polynucleotide constructs encoding for such polypeptide constructs, vectors and cells comprising such polynucleotide and/or polypeptide constructs, production methods, compositions comprising a plurality of such polypeptide-containing EVs, as well as medical applications of such EVs and pharmaceutical compositions containing such EVs.

For convenience and clarity, certain terms employed herein are collected and described below. Unless otherwise defined, all technical and scientific terms used herein have the same meaning as commonly understood by one of ordinary skill in the art to which this invention belongs.

Where features, aspects, embodiments, or alternatives of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects and/or embodiments of the invention. For example, the various at least one polypeptides of interest (PoI) described in connection with the EVs is to be understood to be disclosed and relevant also in the context of the polypeptide constructs or in the context of the pharmaceutical compositions comprising EVs, or as expression products of the polynucleotide constructs as per the present invention. Furthermore, certain embodiments described in connection with certain aspects, for instance the administration routes of the EVs, as described in relation to aspects pertaining to treating certain medical indications, may naturally also be relevant in connection with other aspects and/or embodiment such as aspects/embodiments pertaining to the pharmaceutical compositions or the intracellular delivery methods of the present invention. As a general remark, the proteins of interest (POIs), the exosomal sorting domains (interchangeably termed e.g. “EV sorting domains” or “exosomal polypeptides” or “EV proteins” or “EV polypeptides”), the multimerization domains, and the targeting moieties, the cell sources, and all other aspects, embodiments, and alternatives in accordance with the present invention may be freely combined in any and all possible combinations without deviating from the scope and the gist of the invention. Furthermore, any polypeptide or polynucleotide or any polypeptide or polynucleotide sequences (amino acid sequences or nucleotide sequences, respectively) of the present invention may deviate considerably from the original polypeptides, polynucleotides and sequences as long as any given molecule retains the ability to carry out the technical effect associated therewith. As long as their biological properties are retained the polypeptide and/or polynucleotide sequences according to the present application may deviate with as much as 50% (calculated using for instance BLAST or ClustalW) as compared to the native sequence, although a sequence identity that is as high as possible is preferable. The combination (fusion) of e.g. at least one POI and at least one multimerization domain and at least one exosomal sorting domain implies that certain segments of the respective polypeptides may be replaced and/or modified, meaning that the deviation from the native sequence may be considerable as long as the key properties are conserved. Similar reasoning thus naturally applies to the polynucleotide sequences encoding for such polypeptides.

The term “multimerization domain” may be understood to relate to any polypeptide or protein that enables multimerization (i.e. formation of biomolecular complexes) of several copies of the fusion polypeptide constructs as described herein. In advantageous preferred embodiments, the multimerization domains are homo-multimerization domains, as their primary function is to bring together identical fusion polypeptide constructs in order to increase the efficiency with which such fusion polypeptides are loaded into EVs. In other embodiments, such multimerization domains may be hetero-multimerization domains such as Fos and Jun leucine zipper heterodimer. Some of the preferred homo-multimerization domains of the present invention include the following non-limiting examples: leucine zipper homodimerisation domain of GCN4 from S. cerevisiae, retro-leucine zipper homodimerisation domain of GCN4 from S. cerevisiae, fold-on homotrimerisation domain of fibritin (from the T4 bacteriophage), Fragment X homotetramerisation domain of the phosphoprotein from human respiratory syncytial virus A, human alpha helical coiled coil oligomerisation domain of collagen superfamily, transmembrane homopentameric domain of cardiac phospholamban (human), homodimerization domain of human parathyroid hormone, transmembrane homodimeric domain of human glycophorin A, trimerisation domain of Gp41 (from HIV), C-terminal homodimeric domain of oncoprotein E7, EVH2 homotetramer domain of vasodilator-stimulated phosphoprotein (human), mitochondrial antiviral-signaling protein CARD filament and/or any combination thereof. The multimerization domains herein may be either dimerization domains, trimerization domains, tetramerization domains, or essentially any higher order of multimerization domains, as long as the domain is capable of facilitating interaction between at least two domains (and the polypeptides of which they form part).

The terms “extracellular vesicle” or “EV” or “exosome” shall be understood to relate to any type of vesicle that is, for instance, obtainable from a cell, for instance a microvesicle (e.g. any vesicle shed from the plasma membrane of a cell), an exosome (e.g. any vesicle derived from the endo-lysosomal pathway), an apoptotic body (e.g. obtainable from apoptotic cells), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. Furthermore, the said terms shall also be understood to relate to lipoprotein particles, such as LDL, VLDL, HDL and chylomicrons, as well as extracellular vesicle mimics, cellular membrane vesicles obtained through membrane extrusion or other techniques, etc. Exosomes represent one advantageous type of EVs, but all EVs as mentioned herein are within the scope of the present invention. Essentially, the present invention may relate to any type of lipid-based structure (with vesicular morphology or with any other type of suitable morphology) that can act as a delivery or transport vehicle for the polypeptide construct comprising the POI, a multimerization domain, and an exosomal sorting domain. It will be clear to the skilled artisan that when describing medical and scientific uses and applications of the EVs, the present invention normally relates to a plurality of EVs, i.e. a population of EVs which may comprise thousands, millions, billions or even trillions of EVs. In the same vein, the term “population”, which may e.g. relate to an EV comprising a certain type of POI, shall be understood to encompass a plurality of entities constituting such a population. In other words, individual EVs when present in a plurality constitute an EV population. Thus, naturally, the present invention pertains both to individual EVs comprising various POIs and populations comprising EVs which in turn comprise various POIs, as will be clear to the skilled person.

The terms “exosomal sorting domain”, “EV sorting domain”, “EV sorting protein”, “EV protein”, “EV polypeptide”, “exosomal polypeptide” and “exosomal protein” and similar are used interchangeably herein and shall be understood to relate to any polypeptide that can be utilized to transport a polypeptide construct (which typically comprises, in addition to the exosomal sorting protein, at least one POI and at least one polypeptide-based multimerization domain) to a suitable vesicular structure, i.e. to a suitable EV. More specifically, the term “exosomal sorting domain” shall be understood as comprising any polypeptide that enables transporting, trafficking or shuttling of a polypeptide construct (which as abovementioned typically comprises at least one POI and at least one multimerization domain, but which may also include other types of polypeptide domains) to a vesicular structure, such as an exosome. Typically, such exosomal sorting domains are proteins that are naturally present in EVs, in particular proteins that are naturally present in and/or contribute to the formation of exosomes. Examples of such exosomal sorting domains are for instance CD9 (SEQ ID NO 1), CD53 (SEQ ID NO 2), CD63 (SEQ ID NO 3), CD81 (SEQ ID NO 4), CD54 (SEQ ID NO 5), CD50 (SEQ ID NO 6), FLOT1 (SEQ ID NO 7), FLOT2 (SEQ ID NO 8), CD49d (SEQ ID NO 9), CD71 (SEQ ID NO 10), CD133 (SEQ ID NO 11), CD138 (SEQ ID NO 12), CD235a (SEQ ID NO 13), ALIX (SEQ ID NO 14), Syntenin-1 (SEQ ID NO 15), Syntenin-2 (SEQ ID NO 16), Lamp2b (SEQ ID NO 17), syndecan 1-4 (SEQ ID NOs 72-75), TSG101 (SEQ ID NO 83), HIV Gag p6-1 (SEQ ID NO 86) and numerous other polypeptides capable of transporting a polypeptide construct to an EV are comprised within the scope of the present invention. In certain advantageous embodiments, the exosomal sorting domain is a soluble exosomal polypeptide. Such soluble EV proteins are highly effective at transporting POIs into EVs and with the addition of the multimerization domains of the present invention can be highly active transporters for various POIs into the vesicular (EV) membrane.

The terms “protein of interest”, “polypeptide of interest”, “POI”, “therapeutic polypeptide of interest”, “biotherapeutic”, “biologic”, and “protein biologic” are used interchangeably herein and shall be understood to relate to any polypeptide that can be utilized for therapeutic purposes through e.g. binding a target and/or in any other way interacting with an interaction partner and/or replace a protein and/or supplement or complement an existing intracellular protein, thereby exerting its therapeutic effect. Said terms may represent the following non-limiting examples of therapeutic polypeptides of interest: antibodies, intrabodies, single chain variable fragments (scFv), affibodies, bi-och multispecific antibodies or binders, receptors, decoy receptors, signal transducers, ligands, enzymes for e.g. enzyme replacement therapy or gene editing, tumor suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins (for instance Pseudomonas exotoxins), structural proteins, neurotrophic factors such as NT3/4, brain-derived neurotrophic factor (BDNF) and nerve growth factor (NGF) and its individual subunits such as the 2.5S beta subunit, ion channels, membrane transporters, proteostasis factors, proteins involved in cellular signaling, translation- and transcription related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins, mitochondrial proteins, and heat shock proteins, etc. In one preferred embodiment, the POI is a CRISP R-associated (Cas) polypeptide with intact nuclease activity which is associated with (i.e. carries with it) an RNA strand that enables the Cas polypeptide to carry out its nuclease activity in a target cell once delivered by the EV. Alternatively, the Cas polypeptide may be catalytically inactive, to enable targeted genetic engineering. Yet another alternative may be any other type of CRISPR effector such as the single RNA-guided endonuclease Cpf1. The inclusion of Cpf1 as the PoI is a particular preferred embodiment of the present invention, as it cleaves target DNA via a staggered double-stranded break, Cpf1 may be obtained from species such as Acidaminococcus or Lachnospiraceae. In yet another alternative, the Cas polypeptide may also be fused to a transcriptional activator (such as the P3330 core protein), to specifically induce gene expression. Additional preferred embodiments include POIs selected from the group comprising enzymes for lysosomal storage disorders, for instance glucocerebrosidases such as imiglucerase, alpha-galactosidase, alpha-L-iduronidase, iduronate-2-sulfatase and idursulfase, arylsulfatase, galsulfase, acid-alpha glucosidase, sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alpha-N-acetyl neuraminidase, GlcNAc phosphotransferase, mucolipin1, palmitoyl-protein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2, and hexoaminidase. In other embodiments, the POI may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g. myosin, actin, calcium/binding proteins such as troponin, or structural proteins such as Dystrophin, utrophin, titin, nebulin, dystrophin-associated proteins such as dystrobrevin, syntrophin, syncoilin, desmin, sarcoglycan, dystroglycan, sarcospan, agrin, and/or fukutin. In another preferred embodiment, the POI is a so called decoy receptor, i.e. a protein that “decoys” its target protein and thereby blocking the target protein from exerting a certain effect. Non-limiting examples of decoy receptors include TNF receptors 1 and 2, interleukin receptors such as IL23R, IL17R, or IL1betaR, and in some cases interleukin signal transducers such as gp130, which decoys to the IL6/sIL6R complex and thereby inhibiting or decreasing IL6-mediated signaling, preferably trans-signaling.

The terms “source cell” or “EV source cell” or “parental cell” or “cell source” or “EV-producing cell” or any other similar terminology shall be understood to relate to any type of cell that is capable of producing EVs, e.g. exosomes, under suitable cell culturing conditions. Such conditions may be suspension cell culture or in adherent culture or any in other type of culturing system. Hollow-fiber bioreactors and other types of bioreactors represent highly suitable cell culturing infrastructure. The source cells per the present invention may be select from a wide range of cells and cell lines, for instance mesenchymal stem or stromal cells or fibroblasts (obtainable from e.g. bone marrow, adipose tissue, Wharton's jelly, perinatal tissue, tooth buds, umbilical cord blood, skin tissue, etc.), amnion cells and more specifically amnion epithelial cells optionally expressing various early markers, myeloid suppressor cells. Cell lines of particular interest include human umbilical cord endothelial cells (HUVECs), human embryonic kidney (HEK) cells, endothelial cell lines such as microvascular or lymphatic endothelial cells, chondrocytes, MSCs, airway or alveolar epithelial cells, and various other non-limiting examples of cell sources.

In a first aspect, the instant invention relates to a what is essentially a tri-domain polypeptide construct. Typically, such polypeptide constructs comprise (i) at least one protein of interest (POI), (ii) at least one multimerization domain, and (iii) at least one exosomal sorting domain. The design of the tri-domain polypeptide construct enables highly efficient loading of a POI into an EV, such as an exosome, and also drives increased production of EVs from EV source cells.

The multimerization polypeptide domain is an important component to achieve this increasing loading of the resultant EVs, and such multimerization domains may interestingly be selected from a large variety of different species and may also display relatively different mechanisms of action (e.g. it may be a hetero-dimerization domain, or it may be a homo-trimerization domain, or a homopentameric domain, etc.). However, in preferred embodiments the multimerization domains are homo-multimerization domains, as these enable a simple design of the fusion proteins and importantly supports controlled loading of one single type of fusion polypeptide constructs into EVs (as opposed to multiple fusion constructs). As is evident from the above, the multimerization domains may be either dimerization domains, trimerization domains, tetramerization domains, or essentially any higher order of multimerization domains, as long as the domain is capable of facilitating interaction of at least two domains (and the polypeptides of which they form part). For instance, a non-limiting list of multimerization domains comprises the following domains: leucine zipper homodimerisation domain of GCN4 from S. cerevisiae (SEQ ID NO 18), Retro-Leucine zipper homodimerisation domain of GCN4 from S. cerevisiae (SEQ ID NO 19), Fold-on homotrimerisation domain of Fibritin (from the T4 bacteriophage) (SEQ ID NO 20), Fragment X homotetramerisation domain of Phosphoprotein (from human respiratory syncytial virus A) (SEQ ID NO 21), human alpha helical coiled coil oligomerisation domain of collagen superfamily (SEQ ID NO 22), leucine zipper hetrodimerisation domain of Fos (SEQ ID NO 68) and Jun (human) (SEQ ID NO 23), transmembrane homopentameric domain of Cardiac phospholamban (human) (SEQ ID NO 24), homodimerization domain of parathyroid hormone (human) (SEQ ID NO 25), transmembrane homodimeric domain of Glycophorin A (human) (SEQ ID NO 26), trimerisation domain of Gp41 (from HIV) (SEQ ID NO 27), C-terminal Homodimeric domain of oncoprotein E7 (from HPV 45) (SEQ ID NO 28), and EVH2 homotetramer domain of Vasodilator-stimulated phosphoprotein (human) (SEQ ID NO 29), mitochondrial antiviral-signaling protein CARD filament (SEQ ID NO 85) and/or any combination thereof.

The multimerization domain may be placed in several different locations in the polypeptide construct. For instance, the multimerization domain may be placed between the POI sequence and exosomal sorting domain sequence, within or adjacent to the exosomal sorting domain sequence, and/or within or adjacent to the POI sequence. Overall, the design of the tri-domain polypeptide construct (with regard to both the choice of multimerization domain and its location in the construct, and with regard to the choice of exosomal sorting domain and its location in the construct) is important for determining where in the EVs that the polypeptide ends up after production in an EV source cell. By selecting e.g. a tetraspanin exosomal sorting protein (e.g. CD9, CD63 or CD81) or any other EV membrane protein (such as Lamp2b) it is possible to enrich for the POI on the EV surface. Conversely, selecting an exosomal sorting protein typically present in the EV lumen, such as ALIX or syntenin, enables enriching for the polypeptide construct (and thereby the POI) essentially inside the EV interior. Naturally, the polypeptide constructs may be present simultaneously on the outside and on the inside of the EVs, as well as in the EV membrane. Furthermore, in preferred embodiments, the fusion polypeptide constructs may comprise various types of linkers between the different domains, i.e. between the at least one POI, the at least one multimerization domain, and the at least one exosomal sorting domain. The linker may for instance be a GS (i.e. glycine-serine) linker, i.e. a linker comprising the amino acids glycine and serine, or any other type of suitable linker domain that ensures that the activity of the different domains is not restricted when they are present in a fusion polypeptide construct.

A typical tri-domain fusion polypeptide construct as per the present invention may be described schematically as follows (the below notation is not to be construed as illustrating any C and/or N terminal direction, it is merely meant for illustrative purposes):

### POI-Multimerization Domain-Exosomal Sorting Domain

In a further embodiment, the present invention pertains to polynucleotide constructs encoding the tri-domain polypeptides of the present invention. Such polynucleotide constructs may be present in various types of vectors and expression constructs, for instance plasmids, mini-circles, viruses (integrating or non-integrating) such as adenoviruses, adeno-associated viruses (AAVs), lentiviruses, etc., linear or circular nucleic acids such as linear DNA or RNA, or single or double stranded DNA stretches, or mRNA or modified mRNA, etc. Importantly, such vectors and expression constructs may in various instances be used as therapeutics in their own right. This is particularly relevant in the context of AAVs, adenoviruses, lentiviruses, mRNAs and modified, synthetic mRNAs, which may all be administered directly to a patient. These vectors and/or expression constructs may be inducible and controlled by an external factor such as tetracyclin or doxycycline or any other type of inducer. Furthermore, polynucleotide constructs comprising the polypeptides of the present invention may be present in essentially any type of EV source cell as per the above. Introduction into a source cell (typically a cell culture comprising a suitable EV-producing cell type for production of EVs) may be achieved using a variety of conventional techniques, such as transfection, virus-mediated transformation, electroporation, etc. Transfection may be carried out using conventional transfection reagents such as liposomes, CPPs, cationic lipids or polymers, calcium phosphates, dendrimers, etc. Virus-mediated transduction is also a highly suitable methodology, and may be carried out using conventional virus vectors such as adenoviral or lentiviral vectors. Virus-mediated transduction is particularly relevant when creating stable cell lines for cell banking, i.e. the creation of master cell banks (MCBs) and working cell banks (WCBs) of EV-producing cell sources.

The exosomal sorting domains of the present invention may be selected from any one of the following proteins: CD9 (SEQ ID NO 1), CD53 (SEQ ID NO 2), CD63 (SEQ ID NO 3), CD81 (SEQ ID NO 4), CD54 (SEQ ID NO 5), CD50 (SEQ ID NO 6), FLOT1 (SEQ ID NO 7), FLOT2 (SEQ ID NO 8), CD49d (SEQ ID NO 9), CD71 (SEQ ID NO 10), CD133 (SEQ ID NO 11), CD138 (SEQ ID NO 12), CD235a (SEQ ID NO 13), ALIX (SEQ ID NO 14), Syntenin-1 (SEQ ID NO 15), Syntenin-2 (SEQ ID NO 16), Lamp2b (SEQ ID NO 17), TSPAN8, TSPAN14, CD37, CD82 (SEQ ID NO 77), CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc Receptors, Interleukin receptors, Immunoglobulins, MHC-I or MHC-II components, CD2, CD3 epsilon, CD3 zeta, CD13, CD18, CD19 (SEQ ID NO 79), CD30 (SEQ ID NO 80), CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8 (SEQ ID NO 78), SLIT2, STX3, TCRA, TCRB, TCRD, TCRG, VTI1A, VTI1B, and any combinations thereof.

The protein(s) of interest (POIs) that are loaded into and onto EVs and exosomes as per the present invention may be selected from essentially any group of proteins and/or peptides. The POI may for instance be:
decoy proteins (interchangeably termed decoy receptors) for binding to disease-causing target proteins;
peptides or proteins for inducing endosomal escape, such as HA2;
peptides or proteins for targeting the EV to a tissue or organ or cell type of interest;
a nuclease for binding to and/or cleaving and/or modifying a nucleic acid target;
antibodies and/or intrabodies;
enzymes such as alpha-glucosidase and/or glucocerebrosidase for enzyme replacement therapy;
transport proteins such as NPC1 or cystinosin;
peptides or proteins for optimizing the in vivo behavior of EVs (e.g. their circulation time or immune system recognition), e.g. CD47 and/or CD55 or parts of these proteins;

As will be understood be the person skilled in the art, the present invention enables highly efficient sorting of essentially any protein and/or peptide of interest into and/or onto an EV. Clearly, the POI may be present in its entirety or domains, regions or derivatives of such POIs may be utilized instead. Thus, the following list merely comprises non-limiting exemplary embodiments of the POIs which may be employed herein without deviating from the scope of the present invention:
gp130 (SEQ ID NO 30), TNFR1 (SEQ ID NO 31), TNFR2 (SEQ ID NO 71), IL17 receptor A (SEQ ID NO 32), IL17 receptor B (SEQ ID NO 33), IL17 receptor C (SEQ ID NO 34), IL17 receptor D (SEQ ID NO 35), IL17 receptor E (SEQ ID NO 36), IL22 receptor subunit alpha-1 (SEQ ID NO 70), IL23R (SEQ ID NO 37), IL1 receptor type 1 (SEQ ID NO 38), IL1 receptor type 2 (SEQ ID NO 39), IL12 receptor subunit beta 1 (SEQ ID NO 40), IL12 receptor subunit beta 2 (SEQ ID NO 41), any other decoy receptor or decoy binder which binds to either one of IL1α, IL1β, IL6, the IL6-ILR complex, IL12, IL17, IL23, TNFα, MCP-1, CCL20, complement protein(s), activin, or myostatin;
a targeting peptide or protein, such as an RVG peptide (SEQ ID NO 84), a VSV-G peptide (SEQ ID NO 42), VSV-G protein (SEQ ID NO 43), a p-selectin binding peptide (SEQ ID NO 44), an e-selectin binding peptide (SEQ ID NO 44) and/or any other targeting peptide or protein;
a cell-penetrating peptide (CPP) (e.g. Tat (SEQ ID NO 45), penetratin, TP10, CADY), a peptide with endosomal escape-enhancing properties (e.g. HA2 protein HA2 subunit (SEQ ID NO 46) or the HA2 fusion subunit (SEQ ID NO 82)), or a nuclear localization signal NLS peptide (for instance the sequence PKKKRKV);
a nucleic acid-binding protein such as a transcription factor, or a nuclease such as Cas, Cas9;
a protein for the treatment of a lysosomal storage disorder, for instance glucocerebrosidases such as imiglucerase, alpha-galactosidase, alpha-L-iduronidase, iduronate-2-sulfatase and idursulfase, arylsulfatase, galsulfase, acid-alpha glucosidase, sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alpha-N-acetyl neuraminidase, GlcNAc phosphotransferase, mucolipin1, palmitoyl-protein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2, and hexoaminidase;
antibodies, intrabodies, single chain variable fragments (scFv), affibodies, bi-och multispecific antibodies or binders, receptors, etc;
tumor suppressors such as p53, pVHL, APC, CD95, ST5, YPEL3, ST7, and ST14;
various POIs such as an Fc-binding domain, Lingo-1 (SEQ ID NO 81), NgR1, FC5, a caspase, Fc-fusion proteins, neurite growth inhibitors (Nogo, OMgp, MAG) and neurite growth inhibitors-receptor complex (eg Nogo-NgR1 complex)

As can be seen from the above non-exhaustive list, the POI may be selected from a very broad group of peptide and/or protein agents. The following represents a non-limiting list of some fusion polypeptides of particular interest which display considerable therapeutic activity in various disease models:
SEQ ID NO 47 (hTNFR1-foldon-N-terminal syntenin): Human TNFR1 receptor extracellular part, transmembrane and partial fragment of the cytoplasmic tail (the signaling competent parts of the cytoplasmic tail has been removed) fused to the foldon trimerization domain. The TNF receptor binds its ligand (i.e. TNFalpha) as a trimer and hence the receptor is already primed to bind the ligand when present in a trimeric form, through the presence of the fold-on multimerization domain. The foldon multimerization domain is further fused with an N-terminal domain of Syntenin and also in a separate polypeptide construct to syndecan. The N-terminal domain of Syntenin has binding and interaction sites for key ESCRT and ESCRT accessory proteins such as ALIX. This enhances the sorting of POI into EVs and may also function as a focal point to increase the vesicle production in the genetically engineered EV source cells. EVs comprising this fusion polypeptide, and also the syndecan-based polypeptide construct, efficiently sequesters TNFalpha and thereby decreases inflammation.
SEQ ID NO 48 (hGP130-LZ-N-terminal syntenin): human GP130 extracellular-transmembrane and parts of its cytoplasmic tail (without the signaling domain) are fused to a leucine zipper (LZ) dimerization domain. GP130 exists as dimer on the cell surface and forms a hexameric complex with 2 molecules of IL-6/sIL-6R heterodimeric complex, therefore this forced dimerization will facilitate an increase in ligand binding affinity. The dimerization domain is fused to the N-terminal domain of Syntenin. EVs comprising this fusion polypeptide efficiently sequester the IL6/IL6R complex and reduces inflammation. An identical fusion polypeptide was also designed and tested, but in that construct the exosomal domain syntenin was exchanged for Alix, which also proved to be a highly efficient construct for surface-displaying the gp130 decoy receptor on the exosome surface, resulting in reduced IL6 signaling.
SEQ ID NO 49 (hGP130-fragment X-N-terminal syntenin): human GP130 extracellular-transmembrane and parts of its cytoplasmic tail (without the signaling domain) fused to fragment X tetramerisation domain, in order to increase the loading and to improve receptor affinity towards its ligand (i.e. the complex between IL6 and IL6R. The tetramerisation domain is fused to the N-terminal domain of Syntenin. Another example of a fusion polypeptide construct to enable polypeptide-carrying EVs to inhibit IL6-mediated inflammation.
SEQ ID NO 50 (hGP130-LZ-Transferrin receptor endosomal domain): human GP130 extracellular-transmembrane and parts of its cytoplasmic tail (without the signaling domains) fused to LZ dimerization domain. The LZ domain is fused to a part of the cytoplasmic tail of the transferrin receptor which has domains that direct it to the endosomes (i.e. SEQ ID NO 76, which forms part of the transferrin receptor, also known as CD71 (SEQ ID NO 10)). This will enhance the sorting of the construct to endosomes and subsequently to released EVs (exosomes). Another example of a fusion polypeptide construct to enable polypeptide-carrying EVs to inhibit IL6-mediated inflammation.
SEQ ID NO 51 (P-selectin binding peptide-hGP130-fragment X-N-terminal syntenin): P-selectin binding peptide (which may be used for both targeting to sites of inflammation as well as a way to decrease the lymphocyte infiltration into tissue affected by inflammation) fused to human GP130 extracellular-transmembrane and parts of its cytoplasmic tail (without the signaling domain), fused to fragment X tetramerisation domain (the tetramerisation domain was chosen to increase the loading and for improving receptor affinity towards its ligand). The tetramerisation domain is fused to the N-terminal domain of Syntenin, again to increasing exosomal sorting and to stimulate increased production of exosomes. EVs carrying such fusion polypeptides were shown to target to sites of inflammation, decrease lymphocyte infiltration, and overall reduce inflammation in vivo. CD9 was also evaluated as the exosomal sorting domain and showed modest efficacy when tested using the same fusion polypeptide (i.e. replacing syntenin).
SEQ ID NO 52 (Fragment X-Transferrin receptor-p-selectin binding peptide): P-selectin binding peptide (for both targeting to sites of inflammation as well as a way to decrease the lymphocyte infiltration into tissue affected by inflammation) fused to human transferrin receptor-transmembrane and parts of its cytoplasmic tail fused to fragment X tetramerisation domain (the tetramerisation domain was chosen to increase the loading and for improving receptor affinity towards its ligand).
SEQ ID NO 53 (P-selectin binding peptide-hGp130-LZ-N-terminal syntenin): P-selectin binding peptide (for both targeting to sites of inflammation as well as to decrease the lymphocyte infiltration into tissue affected by inflammation) fused to human GP130 extracellular-transmembrane and parts of its cytoplasmic tail (without the signaling parts) fused to LZ dimerization domain. Again, EVs carrying P-selecting fusion polypeptides showed targeting to sites of inflammation in mice, and reducing overall inflammation.
SEQ ID NO 54 (hTNFR-Z domain-TNFR transmembrane domain-foldon-N-terminal syntenin): Human TNFR1 receptor extracellular part (in the extracellular part the Z-domain has also been introduced. The Z-domain will bind the FC part of antibodies and hence increase the circulation time of the EVs by exploiting the process of opsonisation. Transmembrane and partial fragment of the cytoplasmic tail (the signaling competent parts of the cytoplasmic tail has been removed) are fused to the foldon trimerization (Foldon trimerization domain chosen because the TNF receptor binds the ligand as a trimer and hence the receptor is already primed to bind the ligand). The foldon domain is further fused with the N-terminal domain of Syntenin (the N-terminal domain of Syntenin has binding and interaction sites for some ESCRT and ESCRT accessory proteins such as ALIX. This will increase the sorting into EVs and can also function as a focal point to increase the vesicle production in the genetically engineered cells, in addition to exerting therapeutic effect through binding to TNFalpha thereby reducing inflammatory processes. ALIX has in several experiments been used instead of syntenin, i.e. creating the following fusion protein construct: hTNFR-Z domain-TNFR transmembrane domain-foldon-ALIX.
SEQ ID NO 55 (IL23 receptor without signaling domain-Fos LZ-Syntenin): Human IL23 receptor without the signaling domain fused to Leucine zipper Fos. Fos dimerizes with its partner Jun to form heterodimeric complexes. Since IL23 receptor form heterodimeric complexes with ID 2 receptor subunit beta 1 naturally the IL12 receptor below is equipped with Jun. The IL23-Fos protein is further fused to syntenin and in another fusion polypeptide to syndecan. Both fusion polypeptides achieved dose-dependent reduction of IL23 in vivo in an LPS-induced inflammation model.
SEQ ID 56 (Interleukin-12 receptor subunit beta-1-Jun LZ-Syntenin): Human IL12 receptor subunit beta-1 receptor without the signaling domain fused to Leucine zipper Jun. Jun dimerize with its partner Fos to form heterodimeric complexes. Since ID 2 receptor form heterodimeric complexes with IL23 receptor naturally the IL23 receptor is equipped with Fos. The ID 2-Jun protein is further fused to N-terminal Syntenin.
SEQ ID NO 57 (IL17 C receptor-LZ-N-terminal Syntenin): human IL17 C receptor extracellular-transmembrane and parts of its cytoplasmic tail (without the signaling parts) fused to LZ dimerization domain. The dimerization domain is fused to the N-terminal domain of syntenin. Syndecan has in many experiments been used instead of syntenin, i.e. creating the following fusion protein construct: ID 7C receptor-LZ-syndecan.
SEQ ID NO 58 (CD63-BBK32 FBN BR 2L): Human CD63 with BBK32 as the POI inserted in the second loop. BBK32 is derived from Borrelia bacteria and binds to endothelial cells, which increases the half-life of BBK32-coated exosomes in blood.
SEQ ID NO 59 (DGPSGFP): DGPS is a phosphatidylserine (PS)-binding peptide which is used to coated EVs with a POI, in this case GFP as a model POI.
SEQ ID NO 60 (amino acids 520-610 in VP1 AAV PHP.A-GP130 transmembrane-LZ-N terminal syntenin): Amino acids 520-610 of VP1 of the adeno-associated virus (AAV) capsid (inserted is a 7-mer stretch of amino acids to decrease liver uptake of AAV virus) fused to human GP130 extracellular-transmembrane and parts of its cytoplasmic tail (without the signaling parts) fused to LZ dimerization domain (since GP130 exists as dimer on the cell surface and forms a hexameric complex with 2 molecules of IL-6/sIL-6R heterodimeric complex, therefore this forced dimerization will facilitate in increase in ligand binding affinity). The dimerization domain was fused to the N-terminal domain of syntenin and also to CD63 and CD81, with all construct showing activity and decreased liver uptake.
SEQ ID NO 61 (amino acids 520-610 in VP1 AAV PHP.B-GP130 transmembrane-LZ-N terminal syntenin): Amino acids 520-610 of VP1 of AAV virus capsid (inserted is a 7 mer shown to increase brain uptake of AAV virus, this will therefore increase brain uptake of EVs with this construct) fused to human GP130 extracellular-transmembrane and parts of its cytoplasmic tail (without the signaling parts) fused to LZ dimerization domain (since GP130 exists as dimer on the cell surface and forms a hexameric complex with 2 molecules of IL-6/sIL-6R heterodimeric complex, therefore this forced dimerization will facilitate in increase in ligand binding affinity). The dimerization domain was fused to the N-terminal domain of Syntenin but in separate experiments also to FLOT1 to validate the general applicability of other components of the fusion polypeptides.
SEQ ID NO 62 (CD63 with brain targeting peptide AAV-PHP.B): AAV brain targeting peptide inserted in the second loop of CD63, leading to increased brain uptake of EVs comprising this fusion polypeptide.
SEQ ID NO 63 (CD63 with brain targeting peptide AAV-PHP.A): AAV peptide that decrease liver uptake is inserted in the second loop of CD63, resulting in decreased liver uptake.
SEQ ID NO 64 (Transferrin receptor-amino acids 520-610 in VP1 AAV PHP.B): Human transferrin receptor fused to AAV brain targeting peptide increases brain uptake of EVs carrying said fusion polypeptide.
SEQ ID NO 65 fused to SEQ ID NO 15 (CD47 fused to Syntenin, using a variety of different homo-multimerization domains such as leucine zipper, fold-on and fragment X): CD47 fused to N-terminal Syntenin. Display of multiple copies of CD47 on the surface of EVs, and alternatively multiple copies of CD55 (SEQ ID NO 66), results in increased circulation times of such EVs, enabling a longer window for EVs to exert their therapeutic effects. This fusion polypeptide has successfully been combined into combinatorial EVs comprising other fusion polypeptides, to combine the circulation-enhancing effect of this fusion polypeptide with therapeutic effects of other POIs, e.g. decoy receptors.
SEQ ID NO 67 (IL17 A receptor-LZ-N-terminal Syntenin): human IL17 A receptor extracellular-transmembrane and parts of its cytoplasmic tail (without the signaling parts) fused to LZ dimerization domain. The dimerization domain is fused to the N-terminal domain of syntenin or syndecan.
SEQ ID NO 87 (hTNFR-Z domain-TNFR transmembrane domain-foldon-HIV Gag p6): human TNFR receptor fused to the Z domain and combined with a fold-on domain fused onto the HIV Gag p6 exosomal sorting domain, and in another fusion polypeptide construct also to the exosomal sorting domain CD81. CD81 was not as efficient as HIV Gag p6 in trafficking the TNFR to the EV surface, but nonetheless anti-TNFalpha activity was seen with both fusion polypeptides.
SEQ ID NO 88 (Interleukin-1 receptor type 1-HIV Gag p6): human IL1 receptor type 1 is fused to the HIV Gag p6 exosomal sorting domain, and in another fusion polypeptide construct also to the exosomal sorting domain syndecan. Both HIV Gag p6 and syndecan proved highly efficient at transporting the IL1R POI to the surface of exosomes, exerting considerably anti-IL1 signaling effects in vitro.

In a preferred embodiment, any protease cleavage sites in the amino acid sequences of the POIs, the exosomal sorting domains, and/or the multimerization domains (i.e. essentially anywhere in the fusion polypeptide) are mutated or removed (partially or completely), in order to avoid any proteolytic cleavage of the fusion polypeptides. One particular type of protease cleavage site that is important to mask (through removal or mutation) is cleavage sites recognized by proteases such as PMN elastase, matrix metalloproteinases (MMPs) such as MMP-2, and MMP-13, ADAM17, ADAM 10 and other endopeptidases. Thus, in exemplary embodiments of the present invention the fusion polypeptides comprise mutated protease cleavage sites or alternatively the fusion polypeptide are completely devoid of protease cleavage sites. For instance, removal or mutation of an IENVK stretch (residues 198-203) of human TNFR1 provides resistance against ADAM 17 cleavage, and thereby enhances the loading efficiency into EVs and the resulting therapeutic effect. Removal or mutation of protease cleavage sites have been shown to enable increasing the therapeutic activity in vitro and in vivo by at least a factor 2, occasionally with as much as a factor 5 or 10, so this strategy is applied consistently to stabilize the fusion polypeptides.

In another preferred embodiment, the exosomal sorting domains combined with the at least one multimerization domain may contribute to increase the release of EVs from the EV cell source. Without wishing to be bound by any theory, it is surmised that this increase in EV production by the parental cell results from the interaction of the exosomal sorting domains with the ESCRT sorting system. The multimerization domain is likely increasing the interaction points with the ESCRT complexes and thus further increase the vesicle (e.g. exosome) production in the EV source cells. The exosomal sorting domain will interact with the ESCRT components and induce vesicle formation and hence increased vesicle release. The multimerization domains brings several exosomal sorting domains (such as syntenin, syndecan, CD63, CD81, CD133, etc.) into close proximity to each other and thus increase the interaction with ESCRT components such as ALIX that further drives the induction of vesicle formation. Utilizing this approach the vesicle release may be increase by as much as 10-fold when compared with the production of EVs from cells genetically modified to comprise the corresponding construct comprising only the exosomal sorting domain and the protein of interest. Thus, again without wishing to be bound by any particular theory, it is clear that the genetic engineering of EV source cells with polynucleotide constructs encoding polypeptide constructs comprising the novel combination at least one exosomal sorting domain, at least one multimerization domain, and at least one protein of interest is resulting not only in highly therapeutically potent EVs but also a considerably increased EV yield.

The source cells per the present invention may be select from a wide range of cells, for instance mesenchymal stem or stromal cells or fibroblasts (obtainable from e.g. bone marrow, adipose tissue, Wharton's jelly, perinatal tissue, tooth buds, umbilical cord blood, skin tissue, etc.), amnion cells and more specifically amnion epithelial cells, myeloid suppressor cells. Generally, both primary cells and cell lines are suitable sources of exosomes and EVs. Non-limiting examples include for instance the following: human embryonic kidney (HEK) cells, pericytes, endothelial cells, lymphocytes, endothelial cells and epithelial cells from different organs such as from trachea, lung, GI-tract, urinary tract, etc., dendritic cells (DCs) or other cells from the hematopoietic system such as macrophages, monocytes, B- or T-cells, NK cells, neutrophils, eosinophils, mast cells or basophils, erythrocytes or erythrocyte progenitor cells, thrombocytes and megakaryocytes, etc., cells from different origins such as placenta-derived cells (e.g. decidul placenta cells), syncytiotrophoblasts and amniotic epithelial cells, etc., and cells from CNS and PNS such as microglia, astrocytes, oligodendrocytes and Schwann cells, ependymal cells and nerve cells etc., adipocyte cells from brown or white fat, muscle cells of both smooth muscle and skeletal muscle origin as well as heart muscle cells, to name a few. Generally, EVs and exosomes may be derived from essentially any cell source, be it a primary cell source or cell line. The EV source cells may be any embryonic, fetal, and adult somatic stem cell types, including induced pluripotent stem cells (iPSCs) and other stem or progenitor cells derived by any method. When treating neurological diseases, one may contemplate to utilize as source cells e.g. primary neurons, astrocytes, oligodendrocytes, microglia, and neural progenitor cells. The source cell may be either allogeneic, autologous, or even xenogeneic in nature to the patient to be treated, i.e. the cells may be from the patient himself or from an unrelated, matched or unmatched donor. In certain contexts, allogeneic cells may be preferable from a medical standpoint, as they could provide immuno-modulatory effects that may not be obtainable from autologous cells of a patient suffering from a certain indication.

In yet another aspect, the present invention pertains to pharmaceutical compositions comprising EVs in accordance with the present invention. Typically, the pharmaceutical compositions as per the present invention comprise at least one type of therapeutic EV (i.e. a population of EVs comprising a polypeptide construct comprising at least one desired POI) formulated with at least one pharmaceutically acceptable excipient. The at least one pharmaceutically acceptable excipient may be selected from the group comprising any pharmaceutically acceptable material, composition or vehicle, for instance a solid or liquid filler, a diluent, an excipient, a carrier, a solvent or an encapsulating material, which may be involved in e.g. suspending, maintaining the activity of or carrying or transporting the therapeutic delivery vesicles from one organ, or portion of the body, to another organ, or portion of the body (e.g. from the blood to any tissue and/or organ and/or body part of interest). One particularly suitable pharmaceutically acceptable and potentially active excipient is heparin or any of its analogues and/or derivatives. Heparin may be used to increase the half-life and efficacy of the EVs as per the present invention, in part by reducing the liver of uptake of EVs. The EV populations used for in vivo experiments as described herein were normally formulated in liquid formulation, which are primarily based on HEPES buffer comprising suitable additives. Other salt and/or sugar containing solutions have also been used as pharmaceutical compositions comprising the EVs as per the present invention.

The present invention also relates to cosmetic applications of the EVs comprising POIs. Thus, the present invention pertains also to skin care products such as creams, lotions, gels, emulsions, ointments, pastes, powders, liniments, sunscreens, shampoos, etc., comprising a suitable EV, in order to improve and/or alleviate symptoms and problems such as dry skin, wrinkles, folds, ridges, and/or skin creases. In one embodiment of both cosmetic and therapeutic nature, the EVs as per the present invention may comprise a botulinum toxin (e.g. botox, for instance botulinum toxin types A-G) as the PoI (botulinum toxins may not necessarily be used only for cosmetic applications but could also be applied for e.g. treatment of migraine headaches and dystonia). In a preferred embodiment, EVs (which comprise a at least one type of POI) obtainable from a suitable exosome-producing cell with regenerative properties (such as a mesenchymal stem cell or an amnion epithelial cell) are comprised in a cosmetic cream, lotion, or gel for use in the cosmetic or therapeutic alleviation of wrinkles, lines, folds, ridges and/or skin creases.

In yet another aspect, the present invention relates to EVs as per the present invention for use in medicine. Naturally, when an EV is used in medicine, it is in fact normally a population of EVs that is being used, typically in the form of a pharmaceutical composition comprising the EV population and some form of pharmaceutically acceptable carrier. The dose of EVs administered to a patient will depend on the amount of POI that has been loaded into the EV, the disease or the symptoms to be treated or alleviated, the administration route, the pharmacological action of the POI itself, as well as various other parameters of relevance. The EVs of the present invention may be used for prophylactic and/or therapeutic purposes, e.g. for use in the prophylaxis and/or treatment and/or alleviation of various diseases and disorders. A non-limiting sample of diseases wherein the EVs as per the present invention may be applied comprises Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barré syndrome, acute myocardial infarction, ARDS, sepsis, meningitis, encephalitis, liver failure, kidney failure, graft-vs-host disease, Duchenne muscular dystrophy and other muscular dystrophies, lysosomal storage diseases such as Alpha-mannosidosis, Beta-mannosidosis, Aspartylglucosaminuria, Cholesteryl Ester Storage Disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis Type I, GM1 Gangliosidosis Type II, GM1 Gangliosidosis Type III, GM2—Sandhoff disease, GM2—Tay-Sachs disease, GM2—Gangliosidosis, AB variant, Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II—Hunter Syndrome, MPS IIIA—Sanfilippo Syndrome Type A, MPS IIIB—Sanfilippo Syndrome Type B, MPS IIIB—Sanfilippo Syndrome Type C, MPS IIIB—Sanfilippo Syndrome Type D, MPS IV—Morquio Type A, MPS IV—Morquio Type B, MPS IX—Hyaluronidase Deficiency, MPS VI—Maroteaux-Lamy, MPS VII—Sly Syndrome, Mucolipidosis I—Sialidosis, Mucolipidosis IIIC, Mucolipidosis Type IV, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Neuronal Ceroid Lipofuscinosis T9, Neuronal Ceroid Lipofuscinosis T10, Niemann-Pick Disease Type A, Niemann-Pick Disease Type B, Niemann-Pick Disease Type C, Pompe Disease, Pycnodysostosis, Salla Disease, Schindler Disease and Wolman Disease. etc., neurodegenerative diseases including Alzheimer's disease, Parkinson's disease, Huntington's disease and other trinucleotide repeat-related diseases, dementia, ALS, cancer-induced cachexia, anorexia, diabetes mellitus type 2, and various cancers.

Virtually all types of cancer are relevant targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral, Cavity Cancer, Liposarcoma, Liver Cancer (Primary), Lung Cancer (Non-Small Cell, Small Cell), Lymphomas ((AIDS-related lymphoma, Burkitt lymphoma, cutaneous T-Cell lymphoma, Hodgkin lymphoma, Non-Hodgkin (an old classification of all lymphomas except Hodgkin's) lymphoma, Primary Central Nervous System lymphoma)), Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sezary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumor.

The EVs as per the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes.

In a further aspect, the present invention relates to, as above-mentioned, a method of producing EVs (or more broadly producing populations of EVs) comprising the steps of (a) introducing into a cell source (typically a cell line) one or more polynucleotide construct(s) encoding at least one polypeptide as per the present invention (i.e. a polypeptide comprising at least one exosomal sorting domain, at least one multimerization domain, and at least one POI, i.e. the tri-domain polypeptides disclosed herein), (b) expressing from the polynucleotide construct(s) the polypeptide construct(s) encoded by it, and (c) collecting EVs produced by the cell. Harvesting and purification of EVs may be carried out using a variety of suitable methods, for instance tangential flow filtration (TFF), ultrafiltration, size exclusion chromatography, bead-elute chromatography, or essentially any suitable combination thereof, e.g. a sequential combination of ultrafiltration with bead-elute chromatography.

It shall be understood that the above described exemplifying aspects, embodiments, alternatives, and variants can be modified without departing from the scope of the invention. The invention will now be further exemplified with the enclosed examples, which naturally also may be modified considerably without departing from the scope and the gist of the invention.

### Example 1: Increased Efficiency of TNFR1 Decoy Receptor Exosomes

HEK293T cells stably expressing a luciferase reporter for NfKb-activation was induced with TNF-alpha. Amnion-derived EV source cells were stably transfected with polynucleotide constructs encoding for different fusion polypeptides with different TNFR1-decoy receptors (i.e. the protein of interest, POI), which bind to and “decoy” TNFalpha. EVs comprising the different fusion polypeptides were added to the HEK293T cell culture to evaluate their activity in blocking TNFalpha-mediated signaling.

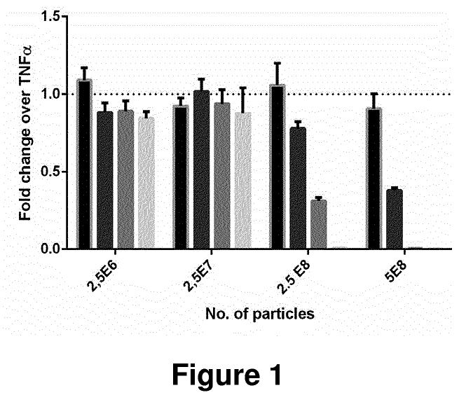

The luciferase signal (a measurement of TNF-alpha activation) was measured 24 hours after treatment. FIG. 1 clearly shows that the multimerization domain, fold-on (SEQ ID NO 48), increases the effect of the EVs (compared to EVs comprising merely a fusion polypeptide consisting of an exosomal sorting domain and the POI) in a dose-dependent manner. In FIG. 1, the black bar with grey border depicts Mock EVs, black bar represents TNFR-syntenin EVs, light grey bar is TNFR-Foldon extracellular-syntenin EVs and dark grey is TNFR-Foldon intracellular-syntenin EVs. As can be seen from FIG. 1, the extracellular fold-on multimerization domain is resulting in an almost complete abrogation of the TNFalpha-mediated signaling.

### Example 2: Increased Efficiency of Gp130 Decoy Receptor EVs after the Insertion of a Multimerization Domain in the Fusion Polypeptide

HeLa cells that stably express a reporter for IL6 activation was treated with hyper-IL6 and EVs (obtained from bone marrow-derived mesenchymal stromal cells) equipped with a gp130 decoy receptor (i.e. the POI (SEQ ID NO 49)) on the external surface of the EVs was added. The IL6 activation was measured 24 hours after the induction. The EVs equipped with the GP130-LZ-N-terminal syntenin construct is clearly better than the EVs only equipped with GP130-N-terminal syntenin at inhibiting IL6-mediated signaling. This highlights the increased activity of EVs comprising with decoy receptor fusion polypeptide into which a multimerization domain has been inserted.

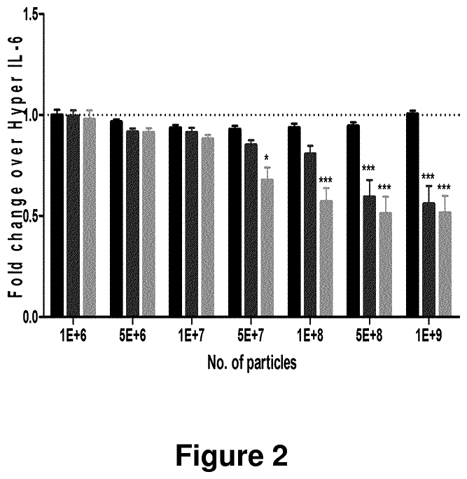

Black bars in FIG. 2 represent mock EVs, dark gray bar depicts EVs comprising regular syntenin-gp130 fusion polypeptides and light gray bar is represents EVs comprising fusion polypeptides comprising a multimerization domain in the form of a leucine zipper (the full fusion polypeptide is gp130-Leucine zipper-syntenin (SEQ ID NO 49)).

### Example 3: LPS-Induced Body Wide Inflammation Efficiently Treated with Gp130 Decoy EVs

Mice received LPS to induce a septic-like condition and after the induction the animals were injected with MSC-derived EVs comprising a fusion polypeptide comprising either (i) an exosomal sorting domain and a POI only, i.e. GP130-N-terminal syntenin construct, or (ii) an exosomal sorting domain, the multimerization domain fragment X (SEQ ID NO XX 21), and with the POI being either GP130 (to decoy the IL6/sILR complex and thus inhibit IL6-mediated signaling) or TNFR (to decoy TNFalpha and block downstream signaling). Both decoy EVs displayed higher activity than mock treated mice, however the decoy EVs equipped with a multimerization domain was better compared to the GP130-N-terminal syntenin EVs (i.e. EVs comprising fusion polypeptides without multimerization domains) and the treatment with the decoy receptor exosomes resulted in 100% survival at the end of the study at 72 hours.

### Example 4: NTA Data Showing Increased Particle Release from Cells Stably Expressing a Multimerization Domain Combined with an Exosomal Sorting Domain

HUVECs were stably transduced by a virus encoding for the TNFR1-foldon-N-terminal syntenin fusion polypeptide construct. Control cells and cells producing EVs comprising the TNFR1-foldon-N-terminal syntenin fusion polypeptide were seeded in 15 cm plates and grown in full serum media for 24 hours. After 24 hours, the media was changed to serum free OptiMEM media and the cells was incubated with OptiMEM for 48 hours. The media was harvested and EVs purified from the media and the subsequent purified EVs were analysed by NTA.

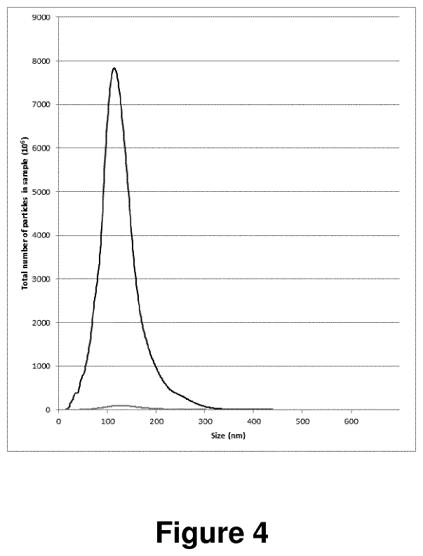

FIG. 4 illustrates that the multimerization domain induce EV release at a considerably higher level than in control cells, as evidenced by the 30-fold increase in exosome production from the cells comprising the polynucleotide encoding for the TNFR1-Foldon-Syntenin fusion polypeptide present on the exosomes (dark line), as compared to the light line depicting exosomes produced by the control cells.

### Example 5: The 2G12 IgG Homodimer Domain Increases Anti-IL6 Trans-Signaling Blocking Activity of Gp130 Decoy Receptor Exosomes

HeLa cells that stably express a reporter for IL6 activation was treated with hyper-IL6 and EVs (obtained from bone marrow-derived MSCs) equipped with a gp130 decoy receptor. IL6 activation was measured 24 hours after the induction. The exosomes comprising the GP130-2G12 IgG homodimer domain-ALIX construct exhibited stronger anti-IL6 trans-signaling activity than the exosomes only equipped with GP130-ALIX only. This highlights the increased activity of EVs comprising with decoy receptor fusion polypeptide into which a multimerization domain has been inserted.

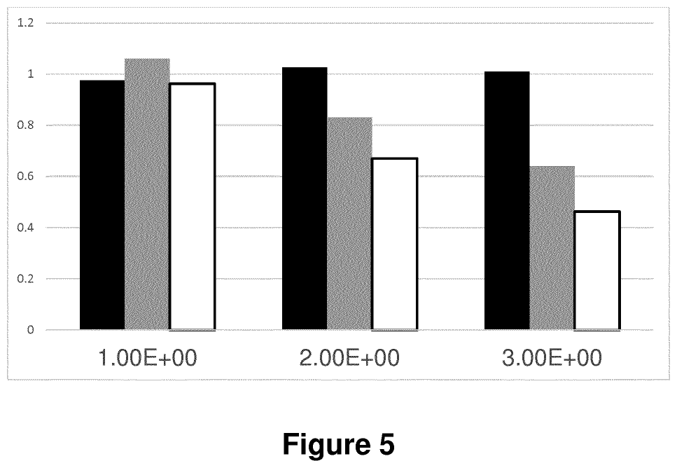

Black bars in FIG. 5 represent mock EVs, gray bar depicts EVs comprising regular ALIX-gp130 fusion polypeptides and white bar with black border represents EVs comprising fusion polypeptides comprising a multimerization domain in the form of a 2G12 IgG homodimer domain (the full fusion polypeptide is gp130-2G12 IgG homodimer domain-ALIX).

### Example 6: The Cardiac Phospholamban Transmembrane Pentamer Increases Expression of the Gaussia Luciferase when Combined with the Tetraspanins CD63 and CD81

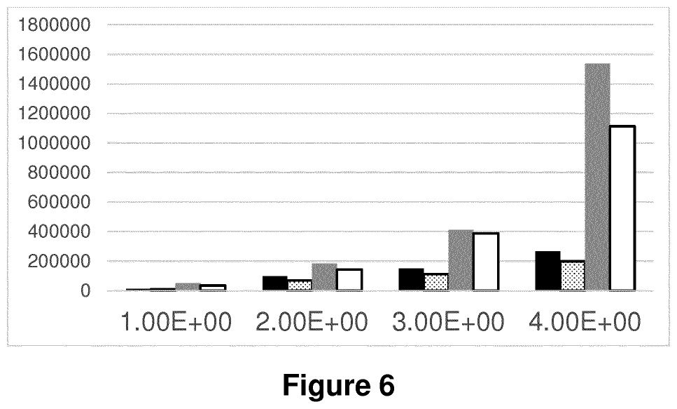

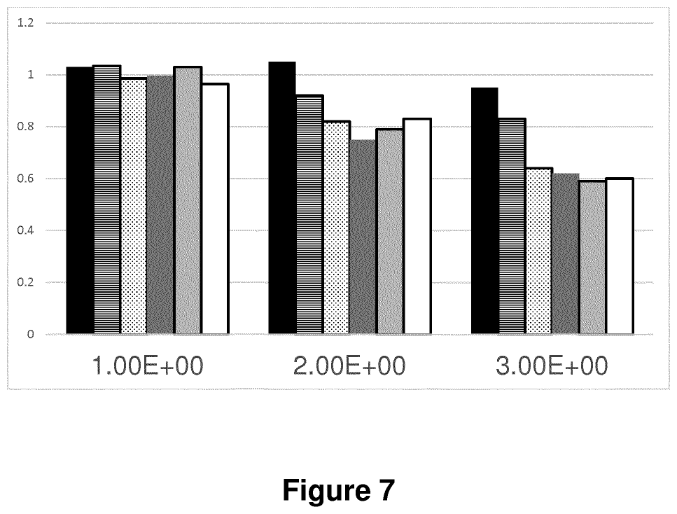

Adipose tissue-derived MSCs were stably transduced with four different Gaussia reporter constructs. Gaussia was fused with CD63 and CD81 with and without multimerization domains. EVs were harvested from conditioned media (incubated for 48 h) and purified with tangential flow and Capto-core liquid chromatography columns. The Gaussia luciferase signal was measured on the harvested EVs as a measurement of CD63/81 loading. As can clearly be seen from FIG. 6, the CD63/81 constructs with the cardiac phospholamban transmembrane pentamer domain has a higher signal and thus increased loading of the EV protein. The black bars in FIG. 7 represent CD63-Gaussia EVs, dotted bar with black borders depicts CD81-Gaussia fusion polypeptides EVs, gray bar represents EVs comprising fusion polypeptides comprising CD63, Gaussia and a multimerization domain in the form of a Cardiac phospholamban transmembrane pentamer multimerization domain (the full fusion polypeptide is CD63-Cardiac phospholamban transmembrane pentamer multimerization domain-Gaussia) and white bar with black border represents CD81-Cardiac phospholamban transmembrane pentamer multimerization domain-Gaussia EVs.

### Example 7: The Leucine Zipper Homodimer Domain Increases Anti-IL6 Trans-Signaling Blocking Activity of Gp130-Displaying Decoy Receptor Exosomes

HeLa cells that stably express a reporter for IL6 activation was treated with hyper-IL6 and with EVs (obtained from bone marrow-derived mesenchymal stromal cells) equipped with a fusion protein construct comprising the gp130 decoy receptor as the POI, the leucine zipper homodomain as the multimerization domain, and various exosomal sorting domains. Black bars in FIG. 7 represent mock EVs, bar with lines and black border depicts EVs comprising regular syntenin-gp130 fusion polypeptides, dotted bar with black border represents EVs comprising fusion polypeptides comprising a multimerization domain in the form of a leucine zipper (the full fusion polypeptide is gp130-Leucine zipper-syntenin), dark grey bar represents Gp130-Leucine zipper-CD63, Light grey bar with black border depicts Gp130-Leucine zipper-CD81 and white bar with black border shows Gp130-Leucine zipper-transferrin receptors endosomal sorting domain. Fusion proteins comprising the leucine zipper domain is clearly better than the EVs only equipped with GP130-syndecan at inhibiting IL6-mediated signaling and this applies across all exosomal sorting domains evaluated.

1. A fusion polypeptide comprising at least one protein of interest (POI), at least one exosomal sorting domain, wherein the exosomal sorting domain is an exosomal protein, exosomal polypeptide or any region, domain, derivative and/or combination of, and at least one homo-multimerization domain.

2. The fusion polypeptide according to claim 1, wherein the homo-multimerization domain is a homo-dimerization domain, a homo-trimerization domain, a homo-tetramerization domain, or any higher order of homo-multimerization domain.

3. The fusion polypeptide according to claim 1, wherein the exosomal sorting domain is selected from the group comprising CD9, CD53, CD63, CD81, CD54, CD50, FLOT1, FLOT2, CD49d, CD71, CD133, CD138, CD235a, transferrin receptor, transferrin receptor endosomal domain, ALIX, syntenin-1 (syntenin), Syntenin-2, Lamp2b, and any region, domain, derivative and/or combination thereof.

4. The fusion polypeptide according to claim 1, wherein the homo-multimerization domain is selected from the group comprising: leucine zipper, foldon domain, fragment X, collagen domain, 2G12 IgG homodimer, mitochondrial antiviral-signaling protein CARD filament, Cardiac phospholamban transmembrane pentamer, parathyroid hormone dimerization domain, Glycophorin A transmembrane, HIV Gp41 trimerisation domain, HPV45 oncoprotein E7 C-terminal dimer domain, and any combination thereof.

5. (canceled)

6. The fusion polypeptide according to claim 1, wherein the POI is selected from at least one of the following groups:
i. gp130, TNFR, IL17R, IL23R, IL1betaR, IL6R, CD55, IL12R, CCR6, any other decoy receptor or decoy binder which binds to either one of IL1α, IL1β, IL6, the IL6-ILR complex, IL12, IL17, IL23, TNFα, MCP-1, CCL20, complement protein(s), activin, or myostatin;
ii. a targeting peptide or protein, such as an RVG peptide, a VSV peptide, a p-selecting binding peptide, an e-selectin binding peptide and/or any other targeting peptide or protein;
iii. a protein for the treatment of a lysosomal storage disorder.

7. The fusion polypeptide according to claim 1, wherein the POI is selected from the group comprising SEQ ID NOs 47-67 and SEQ ID NO 87-88.

8. A polynucleotide construct encoding the fusion polypeptide according to claim 1.

9. An extracellular vesicle (EV) comprising the fusion polypeptide according to claim 1.

10. The EV according to claim 9, wherein the EV comprises a plurality of fusion polypeptides according to claim 1.

11. A cell comprising the fusion polypeptide according to claim 1.

12. A composition comprising a plurality of EVs according to claim 9.

13. A pharmaceutical composition comprising a plurality of EVs according to claim 9 and a pharmaceutically acceptable excipient or diluent.

14. A method for loading a protein of interest (POI) into EVs, comprising the steps of:
i. providing a fusion polynucleotide construct according to claim 7; and,
ii. expressing said fusion construct in an EV-producing cell.

15. An EV comprising the polynucleotide according to claim 8.

16. A cell comprising the polynucleotide construct according to claim 8.

17. A cell comprising the EV according to claim 9.

18. A cell comprising the EV according to claim 15.

19. A composition comprising the fusion polypeptide according to claim 1.

20. A composition comprising the polynucleotide construct according to claim 8.

21. A composition comprising a plurality of EVs according to claim 15.

22. A pharmaceutical composition comprising the fusion polypeptide according to claim 1 and a pharmaceutically acceptable excipient or diluent.

23. A pharmaceutical composition comprising the polynucleotide construct according to claim 8 and a pharmaceutically acceptable excipient or diluent.

24. A pharmaceutical composition comprising a plurality of EVs according to claim 15 and a pharmaceutically acceptable excipient or diluent.
